SEQUENCE LISTING (69 sequences)

SEQ ID NO: 1 - DNA, 3400 bp, Homo sapiens
  exon (1)..(2589)
atggcacatacttttagaggatgctcattggcatttatgtttataatcacgtggctgttgattaaagcaaaaatagatgcgtgcaagagaggcgatgtgactgtgaagccttcccatgtaattttacttggatccactgtcaatattacatgctctttgaagcccagacaaggctgctttcactattccagacgtaacaagttaatcctgtacaagtttgacagaagaatcaattttcaccatggccactccctcaattctcaagtcacaggtcttccccttggtacaaccttgtttgtctgcaaactggcctgtatcaatagtgatgaaattcaaatatgtggagcagagatcttcgttggtgttgctccagaacagcctcaaaatttatcctgcatacagaagggagaacaggggactgtggcctgcacctgggaaagaggacgagacacccacttatacactgagtatactctacagctaagtggaccaaaaaatttaacctggcagaagcaatgtaaagacatttattgtgactatttggactttggaatcaacctcacccctgaatcacctgaatccaatttcacagccaaggttactgctgtcaatagtcttggaagctcctcttcacttccatccacattcacattcttggacatagtgaggcctcttcctccgtgggacattagaatcaaatttcaaaaggcttccgtgagcagatgtaccctttattggagagatgagggactggtactgcttaatcgactcagatatcggcccagtaacagcaggctctggaatatggttaatgttacaaaggccaaaggaagacatgatttgctggatctgaaaccatttacagaatatgaatttcagatttcctctaagctacatctttataagggaagttggagtgattggagtgaatcattgagagcacaaacaccagaagaagagcctactgggatgttagatgtctggtacatgaaacggcacattgactacagtagacaacagatttctcttttctggaagaatctgagtgtctcagaggcaagaggaaaaattctccactatcaggtgaccttgcaggagctgacaggagggaaagccatgacacagaacatcacaggacacacctcctggaccacagtcattcctagaaccggaaattgggctgtggctgtgtctgcagcaaattcaaaaggcagttctctgcccactcgtattaacataatgaacctgtgtgaggcagggttgctggctcctcgccaggtctctgcaaactcagagggcatggacaacattctggtgacttggcagcctcccaggaaagatccctctgctgttcaggagtacgtggtggaatggagagagctccatccagggggtgacacacaggtccctctaaactggctacggagtcgaccctacaatgtgtctgctctgatttcagagaacataaaatcctacatctgttatgaaatccgtgtgtatgcactctcaggggatcaaggaggatgcagctccatcctgggtaactctaagcacaaagcaccactgagtggcccccacattaatgccatcacagaggaaaaggggagcattttaatttcatggaacagcattccagtccaggagcaaatgggctgcctcctccattataggatatactggaaggaacgggactccaactcccagcctcagctctgtgaaattccctacagagtctcccaaaattcacatccaataaacagcctgcagccccgagtgacatatgtcctgtggatgacagctctgacagctgctggtgaaagttcccacggaaatgagagggaattttgtctgcaaggtaaagccaattggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagccagaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctgagtggtgaggcttcaagccttaaagtcagtgtgccctcaaccagcacagcctgccccaattcccccagcccctgctccagcagctgtcatctctgggtgccaccatcggtctggctgcagctagaggacaggcaagccagctctgggggagtcttaggaactgggagttggtcttcactcagatgcctcatcttgcctttcccagggccttaaaattacatccttcactgtgtggacctagagactccaacttgaattcctagtaactttcttggtatgctggccagaaagggaaatgaggaggagagtagaaaccacagctcttagtagtaatggcatacagtctagaggaccattcatgcaatgactatttctaaagcacctgctacacagcaggctgtacacagcagatcagtactgttcaacagaacttcctgagatgatggaaatgttctacctctgcactcactgtccagtacattagacactaggcacattggctgttaatcacttggaatgtgtttagcttgactgaggaattaaattttgattgtaaatttaaatcgccacacatggctagtggctactgtattggagtgcacagctctagatggctcctagattattgagagcctccaaaacaaatcaacctagttctatagatgaagacataaaagacactggtaaacaccaatgtaaaagggcccccaaggtggtcatgactggtctcatttgcagaagtctaagaatgtacctttttctggccgggcgtggtagctcatgcctgtaatcccagcactttgggaggctga

SEQ ID NO: 2 - DNA, 2036 bp, Homo sapiens
  exon (1)..(1989)
atggagccgctggtgacctgggtggtccccctcctcttcctcttcctgctgtccaggcagggcgctgcctgcagaaccagtgagtgctgttttcaggacccgccatatccggatgcagactcaggctcggcctcgggccctagggacctgagatgctatcggatatccagtgatcgttacgagtgctcctggcagtatgagggtcccacagctggggtcagccacttcctgcggtgttgccttagctccgggcgctgctgctacttcgccgccggctcagccaccaggctgcagttctccgaccaggctggggtgtctgtgctgtacactgtcacactctgggtggaatcctgggccaggaaccagacagagaagtctcctgaggtgaccctgcagctctacaactcagttaaatatgagcctcctctgggagacatcaaggtgtccaagttggccgggcagctgcgtatggagtgggagaccccggataaccaggttggtgctgaggtgcagttccggcaccggacacccagcagcccatggaagttgggcgactgcggacctcaggatgatgatactgagtcctgcctctgccccctggagatgaatgtggcccaggaattccagctccgacgacggcagctggggagccaaggaagttcctggagcaagtggagcagccccgtgtgcgttccccctgaaaaccccccacagcctcaggtgagattctcggtggagcagctgggccaggatgggaggaggcggctgaccctgaaagagcagccaacccagctggagcttccagaaggctgtcaagggctggcgcctggcacggaggtcacttaccgactacagctccacatgctgtcctgcccgtgtaaggccaaggccaccaggaccctgcacctggggaagatgccctatctctcgggtgctgcctacaacgtggctgtcatctcctcgaaccaatttggtcctggcctgaaccagacgtggcacattcctgccgacacccacacagaaccagtggctctgaatatcagcgtcggaaccaacgggaccaccatgtattggccagcccgggctcagagcatgacgtattgcattgaatggcagcctgtgggccaggacgggggccttgccacctgcagcctgactgcgccgcaagacccggatccggctggaatggcaacctacagctggagtcgagagtctggggcaatggggcaggaaaagtgttactacattaccatctttgcctctgcgcaccccgagaagctcaccttgtggtctacggtcctgtccacctaccactttgggggcaatgcctcagcagctgggacaccgcaccacgtctcggtgaagaatcatagcttggactctgtgtctgtggactgggcaccatccctgctgagcacctgtcccggcgtcctaaaggagtatgttgtccgctgccgagatgaagacagcaaacaggtgtcagagcatcccgtgcagcccacagagacccaagttaccctcagtggcctgcgggctggtgtagcctacacggtgcaggtgcgagcagacacagcgtggctgaggggtgtctggagccagccccagcgcttcagcatcgaagtgcaggtttctgattggctcatcttcttcgcctccctggggagcttcctgagcatccttctcgtgggcgtccttggctaccttggcctgaacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtgatcgttgaggctcagagagggtgagtgactcgcccgaggctacgtagc

SEQ ID NO: 3 - DNA, 2016 bp, Homo sapiens
  exon (1)..(1470)
atggctctcctctttctcctaccccttgtcatgcagggtgtgagcagggctgagatgggcaccgcggatctggggccgtcctcagtgcctacaccaactaatgttacaattgaatcctataacatgaaccctatcgtatattgggagtaccagatcatgccacaggtccctgtttttaccgtagaggtaaagaactatggtgttaagaattcagaatggattgatgcctgcatcaatatttctcatcattattgtaatatttctgatcatgttggtgatccatcaaattctctttgggtcagagttaaagccagggttggacaaaaagaatctgcctatgcaaagtcagaagaatttgctgtatgccgagatggaaaaattggaccacctaaactggatatcagaaaggaggagaagcaaatcatgattgacatatttcacccttcagtttttgtaaatggagacgagcaggaagtcgattatgatcccgaaactacctgttacattagggtgtacaatgtgtatgtgagaatgaacggaagtgagatccagtataaaatactcacgcagaaggaagatgattgtgacgagattcagtgccagttagcgattccagtatcctcactgaattctcagtactgtgtttcagcagaaggagtcttacatgtgtggggtgttacaactgaaaagtcaaaagaagtttgtattaccattttcaatagcagtataaaaggttctctttggattccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatgagatcagctaagttgcaccaactttgaagtctgattttcctggacagttttctgctttaatttcatgaaaagattatgatctcagaaattgtatcttagttggtatcaaccaaatggagtgacttagtgtacatgaaagcgtaaagaggatgtgtggcattttcacttttggcttgtaaagtacagacttttttttttttttaaacaaaaaaagcattgtaacttatgaacctttacatccagataggttaccagtaacggaacatatccagtactcctggttcctaggtgagcaggtgatgccccagggacctttgtagccacttcactttttttcttttctctgccttggtatagcatatgtgttttgtaagtttatgcatacagtaattttaagtaatttcagaagaaattctcgaagcttttcaaaattggacttaaaatctaattcaaactaatagaattaatggaatatgtaaatagaaacgtgtatattttttatgaaacattacagttagagatttttaaataaagaattttaaaactc

SEQ ID NO: 4 - DNA, 3498 bp, Homo sapiens
  exon (1)..(1626)
atgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttcctcgagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttaatcttcagaaacagtgaacgccaaaaagaactcaagatattctggggactgagcatatgaacctgttcataacaaaggctgtgactcgaaataattaactttgtcaaaatcctgctcacaatttgaagatgaaacttgtcattaggttggcgggaatgagactaaagattgcgctgtgggctgtggtcacgtgctcccagaagacctggaattcaaaagaaatggagctattctttttctccctctttcataactggatgcagctgctcatactcaatcccatattcagcaagtgtgaagctggacgtgatgcaaaataaccgatgccctacaaaaagggcgcatctttaagagttttaatgccagtgcttaattcgaatgaggggattttaagtgtctgaagaggcattttctagggaccagtgggtgactgagtaactgaaatgctgctttcactccctaacaccatggatctggttgtgcataggatgtgggaggaggggctggcagggccgccttcagaggctgcagggcctcagcctcaggatgcatttaatgtatcctggccacagttgcagccaacggttcttgaaagctcggtaaggccctgcaacgcagagcctgcttatgtggatctatttatgggaacttcttaaaaggaccccagaatagctctttatctttcacaagagacacaaattctaattgagttaattatctgggcctttcactttggatgctctgaaacatttgttgattttgtgtgaatgtttatatcaaaatgtttgccaggttgtattagccattgaatagcaaaaaactgatagttacttgcttgttttttaaaaattacatattaaaaatgcccttggcataaggcagcatggtgtggcagttaagagatgggctgtgcagcccatcctgagctccagtcctgagtttgctacttacttctgtggcctctggaaccttatccaacctcttggtgcttcagtttcctcatctgtgaaattagaatttataataattgcacctacctcccaggggtaactaaatgaataaatataataaagtacttacagtggttcctgacacagactcagcactccgtcagtgttgccatgactatttttattatcattattaatgattacttagatcaattatttagcagtggactaatggaagctacagagcagggaagggaagcagatctagggaggaaggcagttttgatttgaggaggtttgcacatgtagagaagcatactggagaagcatatccagagggcgaaagatatctctccattgtgcatctgcctcttttgacgttggaagacacatgtcttactccccaaagggagcccagcactgggagccttcttgatgatctcaaaaataatagctattcaagaaaatcaccaagtgactgtgaaaccgtcagttcggaaggctggttagaacatgtgggagcaacatgaatgttctacaaaagtttaaagcagagattgtttcaaatgggtgtagtagatattactgaaaaccaaaaaagagtgagattgtcagtgtaagaatgtgatttaatgtttgtagtgcttacaattttgtgtaccaactggatgactaaaaagagtaaaataacttaattaatagctcatattttatgtgtgaaaacatgttagtgaacatatataatcaaaatagatttcattgctattgcatagtctctaatacatagaatgattttgcttttctcttttattatacttgctttaaaatacttgaaatatattttgcattaaatgcatttcaagttaaatgtcttaaatgtatacattagatgtgtgttttaaaatgcataaaacacgttgaaa

SEQ ID NO: 5 - DNA, 20 bp, Homo sapiens
ctcctcgaaccaatttggtc

SEQ ID NO: 6 - DNA, 20 bp, Homo sapiens
ctgcacttcgatgctgaagc

SEQ ID NO: 7 - DNA, 21 bp, Homo sapiens
cggagttctataccagagttg

SEQ ID NO: 8 - DNA, 21 bp, Homo sapiens
acatctaacatcccagtaggc

SEQ ID NO: 9 - DNA, 20 bp, Homo sapiens
gatgacagctctgacagctg

SEQ ID NO: 10 - DNA, 19 bp, Homo sapiens
ggcctgatgaccttggatt

SEQ ID NO: 11 - DNA, 20 bp, Homo sapiens
ctgctctgctttgctgaatg

SEQ ID NO: 12 - DNA, 20 bp, Homo sapiens
tcctcttgtgaagacgtggc

SEQ ID NO: 13 - DNA, 20 bp, Homo sapiens
cgtcataccagccattttcc

SEQ ID NO: 14 - DNA, 20 bp, Homo sapiens
ttggtgcaacttagctgatc

SEQ ID NO: 15 - DNA, 18 bp, Homo sapiens
ggcttaaaggggccaagt

SEQ ID NO: 16 - DNA, 20 bp, Homo sapiens
agggagcactatccctcagc

SEQ ID NO: 17 - DNA, 20 bp, Homo sapiens
gtccagagacatgtaagtgc

SEQ ID NO: 18 - DNA, 20 bp, Homo sapiens
gagatgatgcttgtcaacca

SEQ ID NO: 19 - DNA, 20 bp, Homo sapiens
gctagaaagatgaaagctgg

SEQ ID NO: 20 - DNA, 20 bp, Homo sapiens
gtttctgattctggcaactg

SEQ ID NO: 21 - DNA, 30 bp, Homo sapiens
aagaccacaaacacgagagttactaagtga

SEQ ID NO: 22 - protein, 10 aa, Homo sapiens
EPTQARLSES

SEQ ID NO: 23 - protein, 10 aa, Homo sapiens
EPTQAGLSES

SEQ ID NO: 24 - DNA, 30 bp, Homo sapiens
aagaccacaaacacgagggttactaagtga

SEQ ID NO: 25 - DNA, 30 bp, Homo sapiens
ttctggtgtttgtgctcccaatgattcact

SEQ ID NO: 26 - DNA, 19 bp, Homo sapiens
aagtggtcgtcgacagtct

SEQ ID NO: 27 - protein, 6 aa, Homo sapiens
EGAATL

SEQ ID NO: 28 - protein, 6 aa, Homo sapiens
EGVATL

SEQ ID NO: 29 - DNA, 19 bp, Homo sapiens
aagtggttgtcgacagtct

SEQ ID NO: 30 - DNA, 19 bp, Homo sapiens
ttcaccaacagctgtcaga

SEQ ID NO: 31 - DNA, 31 bp, Homo sapiens
gacgatcctcttgtttgtggaaaacgacctt

SEQ ID NO: 32 - DNA, 21 bp, Homo sapiens
ccgaaatggaacgtctgtttt

SEQ ID NO: 33 - protein, 10 aa, Homo sapiens
ALLVFVKQQF

SEQ ID NO: 34 - protein, 7 aa, Homo sapiens
AKGQLCF

SEQ ID NO: 35 - protein, 9 aa, Homo sapiens
SFLCGQLCF

SEQ ID NO: 36 - DNA, 33 bp, Homo sapiens
gacgatcctcttgtttgtgggaacgtctgtttt

SEQ ID NO: 37 - DNA, 33 bp, Homo sapiens
ctgctaggagaacaaacacccttgcagacaaaa

SEQ ID NO: 38 - DNA, 27 bp, Homo sapiens
caacctcgtcccccctacagacttttg

SEQ ID NO: 39 - protein, 9 aa, Homo sapiens
NSCPPHRFV

SEQ ID NO: 40 - protein, 9 aa, Homo sapiens
NSCPPRRFV

SEQ ID NO: 41 - DNA, 27 bp, Homo sapiens
caacctcgtcccccctgcagacttttg

SEQ ID NO: 42 - DNA, 27 bp, Homo sapiens
gttggagcaggggggacgtctgaaaac

SEQ ID NO: 43 - protein, 7 aa, Homo sapiens
WPARAQS

SEQ ID NO: 44 - protein, 7 aa, Homo sapiens
WPAWAQS

SEQ ID NO: 45 - DNA, 24 bp, Homo sapiens
attggccagcccgggctcagagca

SEQ ID NO: 46 - DNA, 24 bp, Homo sapiens
attggccagcctgggctcagagca

SEQ ID NO: 47 - DNA, 24 bp, Homo sapiens
attggccagccygggctcagagca

SEQ ID NO: 48 - protein, 10 aa, Homo sapiens
ARAQSMTYCI

SEQ ID NO: 49 - protein, 10 aa, Homo sapiens
ARAQSTTYCI

SEQ ID NO: 50 - DNA, 30 bp, Homo sapiens
gcccgggctcagagcatgacgtattgcatt

SEQ ID NO: 51 - DNA, 30 bp, Homo sapiens
gcccgggctcagagcacgacgtattgcatt

SEQ ID NO: 52 - DNA, 30 bp, Homo sapiens
gcccgggctcagagcaygacgtattgcatt

SEQ ID NO: 53 - protein, 11 aa, Homo sapiens
PVGQDGGLATC

SEQ ID NO: 54 - protein, 11 aa, Homo sapiens
PVGQDRGLATC

SEQ ID NO: 55 - DNA, 33 bp, Homo sapiens
cctgtgggccaggacgggggccttgccacctgc

SEQ ID NO: 56 - DNA, 33 bp, Homo sapiens
cctgtgggccaggaccggggccttgccacctgc

SEQ ID NO: 57 - DNA, 33 bp, Homo sapiens
cctgtgggccaggacsggggccttgccacctgc

SEQ ID NO: 58 - DNA, 35 bp, Homo sapiens
tcttggtgagaaaagcagacatggctgatatccca

SEQ ID NO: 59 - protein, 11 aa, Homo sapiens
LVRKADMADIP

SEQ ID NO: 60 - protein, 10 aa, Homo sapiens
LVRKDMADIP

SEQ ID NO: 61 - DNA, 32 bp, Homo sapiens
tcttggtgagaaaagacatggctgatatccca

SEQ ID NO: 62 - DNA, 30 bp, Homo sapiens
ctagtggatctacttgtggatgatagcggt

SEQ ID NO: 63 - protein, 10 aa, Homo sapiens
LVDLLVDDSG

SEQ ID NO: 64 - protein, 10 aa, Homo sapiens
LVDLPVDDSG

SEQ ID NO: 65 - DNA, 30 bp, Homo sapiens
ctagtggatctacctgtggatgatagcggt

SEQ ID NO: 66 - DNA, 29 bp, Homo sapiens
tagtggatctacctgtggatgatagcggt

SEQ ID NO: 67 - DNA, 240 bp, Homo sapiens
gtgccacccatgccatctgtgactatatctttttattctttaatagtaactcccttttctaattaaaggtaacaaacaaaacttaaaaaagagatgcccacccaagcgtcattggcatgctgatgttggcaccagtgttgggaagcccttagcatactccaggaagtaggagtgtgtaacgtggggtccctttgtccttcatgcaagggtttcaagagtttagaaaacctatgaaattgc

SEQ ID NO: 68 - DNA, 120 bp, Homo sapiens
caacagttgaggccttacctgccttactggctacaatcactaggaactctctccccaatgtgtaacacaggctaatttctgtctttgacttcagctcttctgacccccaaggggtgacgt

SEQ ID NO: 69 - DNA, 180 bp, Homo sapiens
atgcattgcagaaacaggctcagcttaccctgtgactatgttgccaaggggtcttcacagctttccttctcttttgcagaaagatagagtcttcacggacaagacctcagccacggtcatctgccgcaaaaatgccagcattagcgtgcgggcccaggaccgctactatagctcatcttg

(19) United States
(12) Patent Application Publication
(10) Pub. No.: US 2007/0148642 A1
(43) Pub. Date: Jun. 28, 2007

(54) Method for detecting gene specifying allergic predisposition

(76) Inventors: Naomi Kondo, Aichi (JP); Eiko Matsui, Gifu (JP); Hideo Kaneko, Gifu (JP); Minako Aoki, Gifu (JP); Mizuho Nagao, Gifu (JP); Kimiko Kasahara, Gifu (JP); Koji Tatebayashi, Gifu (JP); Hiroaki Hattori, Saitama (JP); Toru Egashira, Saitama (JP)

Correspondence Address: SUGHRUE MION, PLLC, 2100 PENNSYLVANIA AVENUE, N.W., SUITE 800, WASHINGTON, DC, 20037, US

(21) Appl. No.: 10/526,061

(22) PCT Filed: Aug. 29, 2003
(86) PCT No.: PCT/JP03/11118

§ 371 Date: Jun. 2, 2006

(30) Foreign Application Priority Data
Aug. 30, 2002 (JP) 2002-252446
Sep. 27, 2002 (JP) PCT/JP02/10018

(51) Int. Cl. C12Q 1/68; C07H 21/04

(52) U.S. Cl. 435/6; 536/23.5

(57) ABSTRACT

A method of detecting genes which comprises detecting gene polymorphisms in allergy-predisposition-related genes; i.e., interleukin 12 receptor IL-1 β2 chain and β1 chain genes, interleukin 18 receptor α chain gene, interferon γ receptor 1 chain gene and interleukin 12.p40 subunit gene, to thereby detect allergic predisposition. Use of the present method enables contribution to the prevention of onset of allergic diseases and to the treatment of allergic diseases.

### TECHNICAL FIELD

The present invention relates to a method for detecting a human gene, and more particularly to a method for detecting a gene-polymorphism which can be used as an index for detecting a human allergic predisposition.

### BACKGROUND ART

The term “allergy” is widely used to refer to adverse immunoreaction for living organisms, which occurs in response to invasion of allergens. Allergic reaction is roughly classified into five types (type I through type V) according to the difference in mechanism. The term “atopy” refers to a genetic predisposition which causes, among immunoreactions between antigens and antibodies, type I allergic reaction in which IgE antibody participates. The term “atopic disease” refers to a disease in which atopy is involved. As has been known, most allergic diseases are atopic diseases. Such allergic (atopic) diseases are said to be caused by genetic predispositions and environmental factors (the presence of allergens).

The most essential factors for causing such an allergic (atopic) disease are allergens. Examples of common allergens which cause atopic asthma include house dust, mites, and candida, whereas examples of common allergens which cause allergic rhinitis include cedar pollen and ragweed pollen. Examples of food allergens include egg, milk, and soybean.

An allergic (atopic) predisposition is necessary for the onset of an allergic (atopic) disease. When a living organism having an allergic (atopic) predisposition comes into contact with an allergen, a variety of factors cause an allergic (atopic) disease in the living organism. For example, when an allergen invades a living organism having an allergic (atopic) predisposition, the aforementioned type I allergic reaction, which is known as “immediate-type allergic reaction,” occurs in the living organism, and an IgE antibody is produced therein. The thus-produced IgE antibody is bound, via an Fcε receptor, to mast cells or basophils. When the allergen is bound to the IgE antibody, two molecules of the IgE antibody are cross-linked together, and as a result, a chemical mediator (e.g., histamine, serotonin, heparin, arylsulfatase, NCF (neutrophil chemotactic factor), or ECP (eosinophil cationic protein), which is stored in intracellular granules) is released through degranulation. In addition, a chemical mediator (e.g., leukotriene B₄, C₄, or D₄, prostaglandin E₂, F₂α, or I₂, or thromboxane A₂, which is newly produced from arachidonic acid, or a platelet-activating factor (PAF)) is released. The thus-released chemical mediator such as histamine or leukotriene stimulates inflammatory cells (e.g., eosinophils, neutrophils, lymphocytes, monocytes, or macrophages), and induces smooth muscle constriction, increased vascular permeability, and increased mucous secretion, thereby causing an allergic disease.

As described above, an allergic (atopic) predisposition is necessary for the onset of an allergic (atopic) disease. When a living organism having such a predisposition is sensitized to an allergen, immunoallergic reaction occurs in the living organism, and this reaction and various additional factors cause an allergic (atopic) disease. Furthermore, the allergic (atopic) disease is considered to be affected by an exacerbating factor.

As described above, IgE is the most essential and important protein for inducing allergy.

An object of the present invention is to find a factor for correlating an allergic (atopic) predisposition with a gene relating to production of IgE, and provide means for detecting the allergic (atopic) predisposition, which means employs the factor, and will pave the way for the prevention of the onset of an allergic (atopic) disease or for the treatment of the disease.

### DISCLOSURE OF THE INVENTION

IgE is produced through stimulation of B cells with a cytokine (e.g., interleukin 4 (IL-4)) secreted from Th2 cells (Th2). Thus, IgE production is induced and activated through IL-4-mediated signal transduction.

Meanwhile, production of IgE from B cells by means of IL-4 is suppressed through signal transduction mediated by interferon γ (IFN-γ) secreted from Th1 cells (Th1) Production of IFN-γ is induced through stimulation of the Th1 cells with a cytokine (e.g., interleukin 12 (IL-12) or interleukin 18 (IL-18)), which occurs at the upstream site of the signal transduction pathway.

Thus, production of IgE, which plays an important role in allergic reaction, is controlled by the balance between an IL-4-mediated IgE production promotion system and an IFN-γ-mediated IgE production suppression system. Therefore, IgE production fails to be controlled when these systems are unbalanced. In the IgE production promotion system, IgE production is promoted through binding of IL-4 to IL-4 receptor (IL-4R), whereas in the IgE production suppression system, IgE production is suppressed by means of secretion of IFN-γ, which occurs through binding of IL-12 to IL-12 receptor (IL-12R) or through binding of IL-18 to IL-18 receptor (IL-18R).

The present inventors have considered that a target allergic (atopic) predisposition could be specified at the gene level by focusing on a gene relating to such an IgE production system.

One of the present inventors has measured the amount of IgE produced in subjects on the basis of familial or genetic accumulation often being observed in allergic diseases, and has obtained the result that a proband whose parent (father or mother) exhibits high serum IgE level also exhibits a high IgE level (Naomi Kondo, et al., “Atopic Dermatitis and IL-12 Receptor Gene Mutation,” Clinical Immunology, 36: 535-540, 2001). In another study, peripheral blood mononuclear cells (PBMCs) of a subject were isolated, and the amounts of IFN-γ and IL-4 in the resultant culture supernatant, which were produced through stimulation of PBMCs with a mitogen or an antigen, were measured. The test results revealed that, in the case of a subject exhibiting high IgE level, the IgE level is not positively correlated with the amount of produced IL-4, but is negatively correlated with the amount of produced IFN-γ (Teramoto T., et al., Clin. Exp. Allergy, 28: 74-82, 1998). The results of another study revealed that, in the case of an egg-hypersensitive subject stimulated with ovalbumin, a significant (p<0.01) positive correlation is observed between the levels of IL-4 and serum IgE in the resultant culture supernatant, whereas a significant (p<0.05) negative correlation is observed between the levels of IFN-γ and serum IgE (Kuwabara N, et al., J. Investig. Allergol. Clin. Immunol. 5: 198-204, 1995). The finding that production of IgE through stimulation of PBMCs with IL-4 and a pokeweed mitogen is suppressed by recombinant IFN-γ (Kuwabara N, et al., J. Investig. Allergol. Clin. Immunol. 5: 198-204, 1995) suggests that IFN-γ suppresses IgE production induced by IL-4, and that IgE production increases as IFN-γ production decreases. In addition, the finding that the amount of produced IFN-γ shows a strong positive correlation (r=0.947, n=8) with the amount of measured mRNA of IFN-γ reveals that the aforementioned decrease in IFN-γ production is attributed to a decrease in expression of mRNA of IFN-γ (Teramoto T., et al., Clin. Exp. Allergy, 28: 74-82, 1998).

On the basis of the above-described study results, the present inventors have performed studies on IL-12 and IL-18—which induce IFN-γ production at the upstream site of the signal transduction pathway—in an allergic condition which is considered to be caused by IgE hyperproduction as a result of insufficient IFN-γ production. IL-12 is a 75-kD heterodimeric protein formed of a subunit of 35 kD (p35) and a subunit of 40 kD (p40). IL-12 receptor (IL-12R) is formed of a β1 chain and a β2 chain, and the β2 chain contains three tyrosine residues in an intracellular domain. IL-12R triggers the IL-12 signal cascade. PBMCs derived from a patient were stimulated with IL-12 or IL-18, and the amount of IFN-γ produced in the resultant culture supernatant was measured. As a result, the amount of IFN-γ produced through stimulation with IL-12 or IL-18 was positively correlated with the amount of IL-12 or IL-18, respectively. However, in some cases, the IFN-γ production amount was found to deviate from the positive correlation.

Such results were observed also in the case where PBMCs were stimulated with IL-12 or phytohemagglutinin (PHA); i.e., in some cases, positive correlation was not observed between the IFN-γ production amount and the amount of IL-12 or PHA. In the case where the IFN-γ production amount was found to deviate from the positive correlation, abnormality was observed in the signal transduction system involving a receptor corresponding to IL-12 or PHA.

On the basis of these results, gene analysis was performed. As a result, a plurality of gene polymorphisms relating to allergic (atopic) predispositions were specified (described below).

The present inventors have found that an allergic (atopic) predisposition of a subject can be detected through analysis of such a gene polymorphism, which is strongly related to IgE production balance. The present invention has been accomplished on the basis of this finding.

Accordingly, the present invention provides a gene detection method comprising detecting one or more gene polymorphisms selected from the group consisting of the below-described gene polymorphisms 1 through 13, to thereby detect an allergic predisposition of a subject (hereinafter the method may be referred to as “the present gene detection method”).

As used herein, the term “gene polymorphism” refers to the situation in which there exists a variation at a specific site of the nucleotide sequence of a gene, which variation differs from individual to individual. Firstly, the gene polymorphism encompasses a polymorphism found in a coding region of a specific gene, which polymorphism is specified by amino acid residues encoded by the coding region. This type of polymorphism includes a polymorphism which is not observed in genomic DNA, but is generated in the step in which mRNA is formed from genomic DNA via an mRNA precursor (generally, in the step of splicing of the mRNA precursor), which polymorphism can be generally specified as a polymorphism in cDNA. Secondly, the gene polymorphism encompasses a polymorphism found in a base of a non-coding region (typically, a promoter region, an intron region, etc.) of a specific gene. This type of polymorphism is generally specified by genomic DNA. A gene polymorphism is identified through multilateral analysis of, for example, the polymorphic frequency of the gene, the amount of expression of mRNA of the gene, the amount of an expressed protein, or the function of the protein.

As used herein, the term “wild-type base” or “wild-type amino acid residue” refers to a base or amino acid residue contained in bases or amino acid residues of a polymorphism of a gene, the base or amino acid residue being based on the nucleotide sequence of the gene published in, for example, a database. As used herein, the term “polymorphic base” or “polymorphic amino acid residue” refers to a base or amino acid residue other than the wild-type base or amino acid residue.

As used herein, the expression “detection of a gene polymorphism” refers to the case where, in a sample of a subject, a wild-type base or amino acid residue of a gene polymorphism is detected, or a polymorphic base or amino acid residue of the gene polymorphism is detected.

In the present specification, amino acids are represented by three letter codes or one letter codes as follows: alanine [Ala (by three letter code, the same shall apply hereinafter), A (by one letter code, the same shall apply hereinafter)], valine [Val, V], leucine [Leu, L], isoleucine [Ile, I], proline [Pro, P], phenylalanine [Phe, F], tryptophan [Trp, W], methionine [Met, M], glycine [Gly, G], serine [Ser, S], threonine [Thr, T], cysteine [Cys, C], glutamine [Gln, Q], asparagine [Asn, N], tyrosine [Tyr, Y], lysine [Lys, K], arginine [Arg, R], histidine [His, H], aspartic acid [Asp, D], and glutamic acid [Glu, E].

In the present specification, a gene polymorphism is represented by use of amino acid residues. For example, “A100V” refers to a gene polymorphism specified by alanine (i.e., a wild-type amino acid residue) and valine (i.e., a polymorphic amino acid residue) at position 100 of the amino acid sequence of a target peptide.

In the present specification, a gene polymorphism is also represented by use of the nucleotide sequence of the gene. For example, “1000C/T” refers to a gene polymorphism specified by cytosine (i.e., a wild-type base) and thymine (i.e., a polymorphic base) at position 1,000 of the nucleotide sequence of a target gene.

Unless otherwise specified, wild-type gene sequences employed for specifying the below-described gene polymorphisms 1 through 9 are represented by nucleotide sequence numbers of cDNA, since all the gene polymorphisms 1 through 9 are observed at least in cDNA. The present gene detection method encompasses a gene detection method relating to detection of an allergic predisposition on the basis of gene polymorphisms in genomic DNA or mRNA, the gene polymorphisms corresponding to the polymorphisms observed in cDNA.

Wild-type gene sequences employed for specifying the below-described gene polymorphisms 10 through 13 are represented by use of the nucleotide sequence of an interleukin 12.p40 subunit gene, which is registered in the published database (Gene Bank Accession Number: AY008847).

### BRIEF DESCRIPTION OF THE DRAWINGS

FIG. 1 schematically shows the basic feature of the Invader assay.

FIG. 2 shows the nucleotide sequence, electropherogram, and analysis data in relation to gene polymorphism 1.

FIG. 3 shows the nucleotide sequence, electropherogram, and analysis data in relation to gene polymorphism 2.

FIG. 4 shows the nucleotide sequence, electropherogram, and analysis data in relation to gene polymorphism 3.

FIG. 5 shows the nucleotide sequence, electropherogram, and analysis data in relation to gene polymorphism 4.

FIG. 6 shows The amount of IgE secreted through culturing of peripheral blood mononuclear cells from subjects having gene polymorphisms 1 through 4.

FIG. 7 shows the nucleotide sequence, electropherogram, and analysis data in relation to gene polymorphism 5.

FIG. 8 shows the nucleotide sequence, electropherogram, and analysis data in relation to gene polymorphism 6.

FIG. 9 shows the nucleotide sequence, electropherogram, and analysis data in relation to gene polymorphism 7.

FIG. 10 shows the nucleotide sequence, electropherogram, and analysis data in relation to gene polymorphism 8.

FIG. 11 shows the amount of IgE secreted through culturing of peripheral blood mononuclear cells from subjects having gene polymorphism 8.

FIG. 12 shows the nucleotide sequence, electropherogram, and analysis data in relation to gene polymorphism 9.

FIG. 13 shows the results of pedigree analysis of probands who are found to have gene polymorphism 9.

FIG. 14 shows the nucleotide sequence of a region containing gene polymorphism 10 or 11, and the nucleotide sequence of a region in the vicinity of the polymorphic region.

FIG. 15 shows the nucleotide sequence of a region containing gene polymorphism 12, and the nucleotide sequence of a region in the vicinity of the polymorphic region.

FIG. 16 shows the nucleotide sequence of a region containing gene polymorphism 13, and the nucleotide sequence of a region in the vicinity of the polymorphic region.

### BEST MODE FOR CARRYING OUT THE INVENTION

Embodiments of the present invention will next be described.

As described above, the present invention provides a gene polymorphism detection method comprising detecting a polymorphism of an interleukin 12 receptor (IL-12R) β2 chain gene, an interleukin 12 receptor (IL-12R) β1 chain gene, an interleukin 18 receptor (IL-18R) α chain gene, an interferon γ receptor (IFN-γR) 1 chain gene, or an interleukin 12 (IL-12).p40 subunit gene, which gene relates to an allergic (atopic) predisposition, to thereby detect an allergic (atopic) predisposition.

The IL-12R β2 chain gene and the IL-12R β2 chain protein encoded by the gene have already been analyzed (Presky D, et al., Proc. Natl. Acad. Sci., 93: 14002-14007, 1996). The nucleotide sequence of cDNA of the IL-12R β2 chain gene and the amino acid sequence corresponding to the nucleotide sequence are shown in SEQ ID NO: 1 (Gene Bank Accession Number: U64198). Also, the IL-12R β1 chain gene and the IL-12R β1 chain protein encoded by the gene have already been analyzed (Chua A, et al., J. Immun., 153: 128-136, 1994). The nucleotide sequence of cDNA of the IL-12R β1 chain gene and the amino acid sequence corresponding to the nucleotide sequence are shown in SEQ ID NO: 2 (Gene Bank Accession Number: U03187). Also, the IFN-γR 1 chain gene and the IFN-γR 1 chain protein encoded by the gene have already been analyzed (Aguet M, Cell, 55: 273-280, 1988). The nucleotide sequence of cDNA of the IFN-γR 1 chain gene and the amino acid sequence corresponding to the nucleotide sequence are shown in SEQ ID NO: 3 (Gene Bank Accession Number: J03143). Also, the IL-18R α chain gene and the IL-18R α chain protein encoded by the gene have already been analyzed (Parnet P, J. Biol. Chem., 271: 3967-3970, 1996). The nucleotide sequence of cDNA of the IL-18R α chain gene and the amino acid sequence corresponding to the nucleotide sequence are shown in SEQ ID NO: 4 (Gene Bank Accession Number: U43672). As described above, the nucleotide sequence of genomic DNA of the IL-12.p40 subunit gene has already been analyzed (Gene Bank Accession Number: AY008847).

When specific analysis is performed on the relation between a specific gene polymorphism and an allergic disease (e.g., bronchial asthma, allergic rhinitis, atopic dermatitis, or food allergy) which is considered to be induced by the polymorphic protein encoded by the gene polymorphism, there can be specified gene polymorphisms of the IL-12R β2 chain gene, the IL-12R β1 chain gene, the IL-18R α chain gene, the IFN-γR 1 chain gene, and the IL-12.p40 subunit gene, which polymorphisms can be employed in the present gene detection method. Specifically, when, in patients with an allergic (atopic) disease and healthy human, the polymorphic site and polymorphic frequency of the IL-12R β2 chain gene, IL-12R β1 chain gene, IL-18R α chain gene, IFN-γR 1 chain gene, or IL-12.p40 subunit gene, or the function of a protein encoded by a polymorphism of the gene is analyzed, a target gene polymorphism can be specified. The details of such analysis will be specifically described below in Examples.

As described above, the present inventors have so far detected an allergic-predisposition-related gene polymorphism in the IL-12R β2 chain gene, IL-12R β1 chain gene, IL-18R α chain gene, IFN-γR 1 chain gene, or IL-12.p40 subunit gene. Examples of the gene polymorphism include:

1. a polymorphism of the IL-12R β2 chain gene specified by mutation (or substitution) of a region (or nucleotide) encoding position 313 arginine of the IL-12R β2 chain protein encoded by the gene [e.g., a polymorphism of the gene which is specified such that the position 937 polymorphic base of the IL-12R β2 chain gene is adenine or guanine, and the position 313 polymorphic amino acid residue of the IL-12R β2 chain protein is glycine];

2. a polymorphism of the IL-12R β2 chain gene specified by mutation of a region encoding position 604 alanine of the IL-12R β2 chain protein encoded by the gene [e.g., a polymorphism of the gene which is specified such that the position 1,811 polymorphic base of the IL-12R β2 chain gene is cytosine or thymine, and the position 604 polymorphic amino acid residue of the IL-12R β2 chain protein is valine];

3. a polymorphism of the IL-12R β2 chain gene specified by lack of a region encoding position 619 glycine and subsequent amino acid residues of the IL-12R β2 chain protein encoded by the gene (e.g., a polymorphism of the gene which is specified by lack of bases at positions 1,856 to 1,946 of the IL-12R β2 chain gene);

4. a polymorphism of the IL-12R β2 chain gene specified by mutation of a region encoding position 720 histidine of the IL-12R β2 chain protein encoded by the gene (e.g., a polymorphism of the gene which is specified such that the position 2,159 polymorphic base of the IL-12R β2 chain gene is adenine or guanine, and the position 720 polymorphic amino acid residue of the IL-12R β2 chain protein is arginine];

5. a polymorphism of the IL-12R β1 chain gene specified by mutation of a region encoding position 361 arginine of the IL-12R β1 chain protein encoded by the gene [e.g., a polymorphism of the gene which is specified such that the position 1,081 polymorphic base of the IL-12R β1 chain gene is cytosine or thymine, and the position 361 polymorphic amino acid residue of the IL-12R β1 chain protein is tryptophan];

6. a polymorphism of the IL-12R β1 chain gene specified by mutation of a region encoding position 365 methionine of the IL-12R β1 chain protein encoded by the gene [e.g., a polymorphism of the gene which is specified such that the position 1,094 polymorphic base of the IL-12R β1 chain gene is thymine or cytosine, and the position 365 polymorphic amino acid residue of the IL-12R β1 chain protein is threonine];

7. a polymorphism of the IL-12R β1 chain gene specified by mutation of a region encoding position 378 glycine of the IL-12R β1 chain protein encoded by the gene [e.g., a polymorphism of the gene which is specified such that the position 1,132 polymorphic base of the IL-12R β1 chain gene is guanine or cytosine, and the position 378 polymorphic amino acid residue of the IL-12R β1 chain protein is arginine];

8. a polymorphism of the IL-18R α chain gene which is specified by lack of position 317 alanine of the IL-18R α chain protein encoded by the gene (e.g., a polymorphism of the gene which is specified by lack of bases at positions 950 to 952 of the IL-18R α chain gene);

9. a polymorphism of the IFN-γR 1 chain gene specified by mutation of a region encoding position 467 leucine of the IFN-γR 1 chain protein encoded by the gene [e.g., a polymorphism of the gene which is specified such that the position 1,400 polymorphic base of the IFN-γR 1 chain gene is thymine or cytosine, and the position 467 polymorphic amino acid residue of the IFN-γR 1 chain protein is proline];

10. a polymorphism of the IL-12.p40 subunit gene specified by substitution of position 3,696 guanine by another base, the guanine being in intron 1 of the gene (e.g., a polymorphism of the gene in which the position 3,696 polymorphic base of the IL-12.p40 subunit gene is adenine);

11. a polymorphism of the IL-12.p40 subunit gene specified by substitution of position 3,757 cytosine by another base, the cytosine being in intron 1 of the gene (e.g., a polymorphism of the gene in which the position 3,757 polymorphic base of the IL-12.p40 subunit gene is thymine);

12. a polymorphism of the IL-12.p40 subunit gene specified by substitution of position 12,359 thymine by another base, the thymine being in intron 4 of the gene (e.g., a polymorphism of the gene in which the position 12,359 polymorphic base of the IL-12.p40 subunit gene is guanine); and

13. a polymorphism of the IL-12.p40 subunit gene specified by substitution of position 16,078 cytosine by another base, the cytosine being in intron 6 of the gene (e.g., a polymorphism of the gene in which the position 16,078 polymorphic base of the IL-12.p40 subunit gene is thymine).

As described below, the gene polymorphisms 10 and 11 are in linkage equilibrium with each other in intron 1 of the IL-12.p40 subunit gene. Therefore, when the present gene detection method is performed on intron 1, it is efficient to detect either of the gene polymorphisms 10 and 11. Similar to the case of the gene polymorphisms 10 and 11, the gene polymorphism 12 in intron 4 of the IL-12.p40 subunit gene is in linkage equilibrium with the gene polymorphism 13 in intron 6 thereof. Therefore, when the present gene detection method is performed on intron 4 or 6, it is efficient to detect either of the gene polymorphisms 12 and 13.

A polymorphic site of a gene polymorphism can be detected by means of a generally known method. Examples of the detection method include the RFLP method employing Southern blotting; the PCR-RFLP method; the HET (hetero duplex analysis) method; the DGGE (denaturing gradient gel electrophoresis) method; the DS (direct sequence) method; the CCM (chemical cleavage mismatch) method; the CDI (carbodiimid modification) method; the PCR-SSCP (single-stranded conformation polymorphism) method (hereinafter will be referred to as the “SSCP method”); the PCR/GC-clamp method; and the Invader assay [Third Wave Technologies (US)] [see, for example, Bio Manual Series 1, Idenshi Kogaku no Kiso Gijutsu (“Fundamental Technique of Genetic Engineering”), edited by Tadashi Yamamoto, Yodosha Co., Ltd. (1993), in particular, Myers, R. M., Shefield, V., and Cox, D. R. (1988) in Genomic Analysis: A Practical Approach. K. Davies, ed. IRL Press Limited, Oxford, pp. 95-139 for the PCR/GC-clamp method]. From the viewpoint of simple and correct detection of a gene polymorphism, the Invader assay is preferably selected.

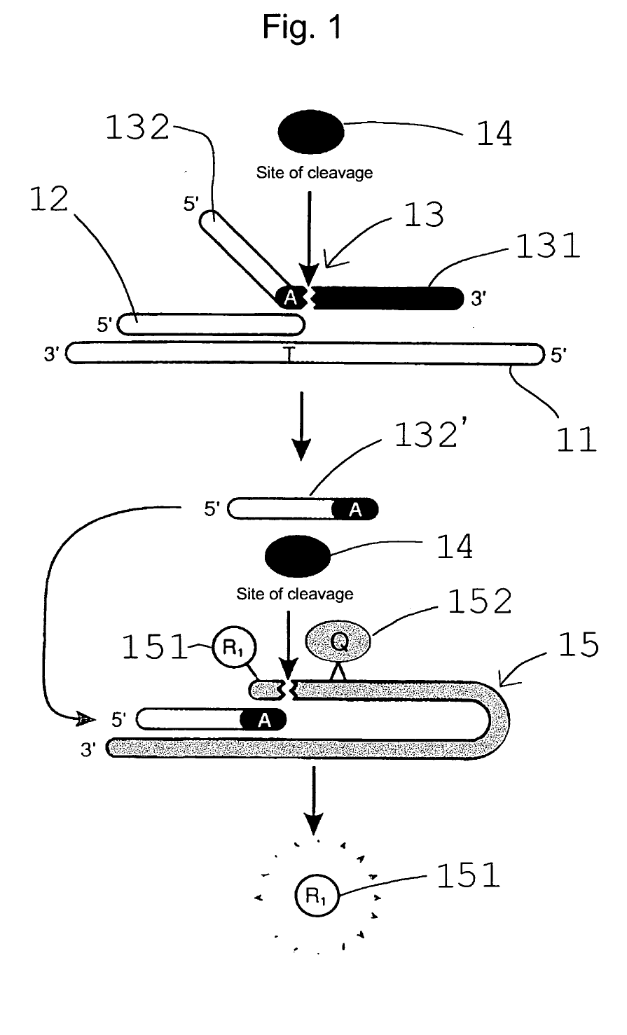

FIG. 1 schematically shows the basic feature of the Invader assay.

As shown in FIG. 1, firstly, a first nucleotide fragment 12 is hybridized with a nucleotide fragment 11 serving as a template [wild-type gene (which may be the nucleotide sequence of genomic DNA or the nucleotide sequence of cDNA)].

The first nucleotide fragment 12—in which the base (A (adenine) in FIG. 1] complementary to the base to be detected [T (thymine) in FIG. 1] of the template nucleotide fragment 11 is located at the 3′-end—is complementary to the template nucleotide fragment 11. (In this case, the base at the 3′-end of the first nucleotide fragment 12 is complementary to the base to be detected. However, even in the case where the 3′-end base is not complementary to the base to be detected, when the 3′-end base interferes in association reaction between the base to be detected and a second nucleotide fragment, a locally triple-stranded structure is formed.)

Subsequently, a second nucleotide fragment 13 is hybridized with the locally double-stranded structure formed of the template nucleotide fragment 11 and the first nucleotide fragment 12.

The second nucleotide fragment 13 is a composite nucleotide fragment including a “complementary portion” 131 which is complementary to the template nucleotide fragment 11, and a “detection portion” 132 which has a detection element and is not complementary to the template nucleotide fragment, wherein the portion 131 is located on the 3′-side, and the portion 132 is located on the 5′-side so as to be continuous with the portion 131. The base located at the 5′-side end of the “complementary portion” 131 is (A) (i.e., a base complementary to the base to be detected (T)).

This second hybridization forms a locally triple-stranded structure including the base to be detected (T) of the template nucleotide fragment 11, the 3′-end base of the first nucleotide fragment 12, and the base (A) located at the 5′-side end of the “complementary portion” 131 of the second nucleotide fragment.

Subsequently, a nuclease 14 having activity to specifically cleave the locally triple-stranded structure on its 3′-side is caused to act on the structure, and a detection portion 132′ of the second nucleotide fragment 13 which has been cleaved by the nuclease [the 3′-end base of the portion 132′ is base (A), which is complementary to the base to be detected (T)] is detected, whereby the template nucleotide fragment 11 can be detected to be a wild-type gene.

As shown in FIG. 1, when a hairpin-shaped probe (nucleotide fragment) 15 labeled with a fluorescent dye 151 in the vicinity of its 5′-end and with a quencher 152 in the vicinity of its 3′-side is caused to coexist with the aforementioned hybridization system, the aforementioned wild-type gene can be detected.

A single-stranded portion (on the 3′-side) of the hairpin-shaped probe 15 is designed so as to be complementary to the detection portion 132 of the second nucleotide fragment 13. The base to be detected (T) is one base on the 5′-side which is adjacent to the base located at the 5′-side end of the single-stranded portion. When the detection portion 132′ is hybridized with the single-stranded portion of the hairpin-shaped probe 15, at the tip of a double-stranded portion of the probe 15, a locally triple-stranded structure is formed of the base (A) located at the 3′-end of the detection portion 132′ and the hairpin-shaped probe 15. The nuclease 14 acts on the locally triple-stranded structure, and the hairpin-shaped probe 15 is cleaved at a site between a portion labeled with the fluorescent dye 151 and a portion labeled with the quencher 152, whereby the portion labeled with the fluorescent dye 151 is released. Since the thus-released portion is no longer affected by the quencher 152, fluorescence emitted from the released portion can be detected. Through detection of the fluorescence, the template nucleotide fragment 11 can be detected to be the wild-type gene in which the base to be detected is not a polymorphic base.

Meanwhile, in the case where the base to be detected of the template nucleotide fragment 11 is not the wild-type base (T) but a single nucleotide polymorphism (SNP) base (e.g., guanine (G)), and the base G is positively detected, the complementary base of the first nucleotide fragment 12 and the second nucleotide fragment 13 is changed from the above-employed A to C (cytosine), which is complementary to G, and the fluorescent dye 151 and the quencher 152 provided on the hairpin-shaped probe 15 are changed to a fluorescent dye which emits fluorescence differing from the above fluorescence and a quencher corresponding to the fluorescent dye, respectively, whereby the SNP base of the template nucleotide fragment 11 can be detected by means of fluorescence emitted from the different fluorescent dye.

When the template nucleotide fragment 11 is a nucleotide fragment including a wild-type base and a polymorphic base; i.e., a hetero-type nucleotide fragment, the nucleotide fragment can be positively detected by means of a mixture of the aforementioned two types of fluorescence.

In the above-described embodiment, the detection system employs the hairpin-shaped probe. However, for example, the detection portion 132 can be directly labeled with a fluorescent dye or an isotope, and the thus-labeled detection portion can be directly detected, whereby a gene polymorphism can be detected. In the above-described embodiment, the base of the template nucleotide fragment is positively detected in both the case where the nucleotide fragment has a polymorphic base and the case where the nucleotide fragment does not have a polymorphic base. However, in either of the above cases, negative detection, in which a label such as fluorescence is not detected, can be performed.

In the above-described Invader assay, as reaction proceeds, the nuclease which specifically cleaves the locally triple-stranded structure continuously acts in a step in which the “detection portion” of the second nucleotide fragment is cleaved, and in a step in which a portion labeled with a fluorophore is separated from a portion labeled with a quencher (in the case where the hairpin-shaped probe is employed). Therefore, a label employed in the Invader assay, such as fluorescence, is sensitized; i.e., the Invader assay involves a very sensitive liquid-phase reaction.

The PCR/GC-clamp method is a modification of the OGGE method (note: DGGE is a method for detecting a substituted base of DNA by means of the difference in mobility between a double-stranded DNA fragment containing the substituted base and a double-stranded DNA fragment containing no substituted base in a polyacrylamide gel with a linear gradient of DNA denaturing agent, which difference is attributed to the difference in the concentration of the DNA denaturing agent in the gel). The PCR/GC-clamp method overcomes the drawback of the DGGE method (i.e., in the case where a DNA fragment contains a plurality of substituted bases, a substituted base in the lowest melting domain of the DNA fragment cannot be detected in a polyacrylamide gel) through addition of a GC-rich fragment (GC-clamp) to the DNA fragment containing the substituted bases to be detected [see, for example, Shefield, V. C. et al. (1989) Proc. Natl. Acad. Sci. USA 86: 232-236].

The PCR/GC-clamp method, whose fundamental operation, etc. are according to the DGGE method, requires a step of adding a GC-clamp to a DNA fragment containing substituted bases to be detected.

In the present gene detection method, no particular limitations are imposed on the source of DNA employed for detection of a polymorphism of the IL-12R β2 chain gene, IL-12R β1 chain gene, IL-18R α chain gene, IFN-γR 1 chain gene, or IL-12.p40 subunit gene, so long as the DNA source is somatic cells of a subject. In the present invention, a blood sample (e.g., peripheral blood cells or leukocytes) is preferably selected.

Specifically, genomic DNA can be extracted, by means of a known technique, from sample cells of a subject (the cells may be those which have been isolated and then cultured), and a polymorphism of a specific gene site (specifically, substitution, deletion, or insertion of a base at the specific gene site) can be detected in the genomic DNA. Alternatively, mRNA can be extracted, by means of a known technique, from sample cells of a subject (the cells may be those which have been isolated and then cultured), and a gene polymorphism can be detected in cDNA obtained by employing the mRNA as a template.

In the case where a polymorphic base is observed at the aforementioned specific gene site through the above detection, when any change in the gene site is correlated with an allergic (atopic) predisposition, there can be specified the type of an allergic (atopic) disease which is highly probable to occur in the subject, or the cause of an allergic (atopic) disease which occurs in the subject.

No particular limitations are imposed on the mode for carrying out the present gene detection method, and the mode can be appropriately selected in accordance with the method for detecting a selected gene polymorphism. Typically, the present gene detection method can be carried out by use of a gene polymorphism detection kit including an element for carrying out the detection method. Specifically, for example, materials and reagents required for carrying out the present gene detection method are added to individual wells of a microplate, and a target gene polymorphism can be detected through reaction for detecting the polymorphism by use of the microplate. Alternatively, detection of a gene polymorphism can be performed more efficiently by use of a microarray instead of a microplate. In the gene polymorphism detection kit, if desired, combinations of elements (e.g., a diluting agent, a reagent, and an instrument for gene detection, such as a microplate or a microarray) can be appropriately determined.

In the case where, for example, a polymorphic base or amino acid residue is detected at a specific gene site of a subject, the subject is determined to have an allergic (atopic) predisposition. Therefore, even when the subject exhibits no abnormality at present, it can be made clear that the subject has high risk for the onset of an allergic (atopic) disease. In such a case, measures for environmental factors (e.g., measures for house dust, or uptake of non-allergic foods) can be taken, to thereby prevent the onset of an allergic (atopic) disease.

Meanwhile, when, for example, the present gene detection method is performed on a subject with an allergic (atopic) disease, the cause of the disease can be assumed at the gene level with high accuracy. In such a case, the allergic (atopic) disease can be treated more effectively by means of a treatment method selected in accordance with the specified genetic abnormality.

Furthermore, elucidating the mechanism by which the genetic abnormality induces the allergic (atopic) disease can pave the way for development of a drug for treating the allergic (atopic) disease.

### EXAMPLES

The present invention will next be described in more detail by way of Examples, which should not be construed as limiting the invention thereto.

[Gene Analysis Method]

Isolation and Culturing of Peripheral Blood Mononuclear Cells

Blood was collected from the vein of a subject into heparin (5 ml), and the blood collected in heparin was layered onto Ficoll (product of Sigma), followed by specific gravity centrifugation, to thereby isolate peripheral blood mononuclear cells. The thus-isolated mononuclear cells were added to an RPMI 1640 medium containing 10% fetal bovine serum, L-glutamine (2 mmol/l), penicillin (100 U/ml), and streptomycin (100 U/ml) such that the amount of the cells became 1×10⁶ cells/ml, followed by culturing in the presence of 5% CO₂. The mononuclear cells were stimulated with phytohemagglutinin (PHA) (product of Gibco BRL, 10 μg/ml), IL-12 (product of R&D, 5 IU/ml), or IL-18 (product of MLB, 400 IU/ml), which was added to the medium.

Quantitative Determination of Cytokine

The thus-stimulated mononuclear cells were cultured for 24 hours, and then collected. Subsequently, cell fractions were removed through centrifugation, to thereby yield a culture supernatant. The amount of IFN-γ contained in the resultant culture supernatant was measured by use of ELISA kit (product of Otsuka Assay).

RNA Extraction and RT-PCR

The mononuclear cells which had been stimulated with PHA and cultured for 24 hours were collected, and RNA was extracted therefrom by use of Isogen kit (product of Nippon Gene Co., Ltd.). Subsequently, cDNA was synthesized from the RNA by means of a customary technique. RT-PCR amplification of cDNA of an IL-12R β1 chain, IL-12R β2 chain, IL-18R α chain, or IFN-γR 1 chain was performed by use of a primer set specific to the chain (described below in Table 1). PCR was performed for 40 cycles, each including 94° C. for one minute, 54° C. for one minute, and 72° C. for one minute.

TABLE 1

| Sequence | Receptor | Primer sequence |
|---|---|---|
| 1 | IL-12R β1 chain | 5′-CTCCTCGAACCAATTTGGTC-3′ |
| 2 | IL-12R β1 chain | 5′-CTGCACTTCGATGCTGAAGC-3′ |
| 3 | IL-12R β2 chain | 5′-CGGAGTTCTATACCAGAGTTG-3′ |
| 4 | TL-12R β2 chain | 5′-ACATCTAACATCCCAGTAGGC-3′ |
| 5 | IL-12R β2 chain | 5′-GATGACAGCTCTGACAGCTG-3′ |
| 6 | IL-12R β2 chain | 5′-GGCCTGATGACCTTGGATT-3′ |
| 7 | IL-18R α chain | 5′-CTGCTCTGCTTTGCTGAATG-3′ |
| 8 | IL-18R α chain | 5′-TCCTCTTGTGAAGACGTGGC-3′ |
| 9 | IFN-γR 1 chain | 5′-CGTCATACCAGCCATTTTCC-3′ |
| 10 | IFN-γR 1 chain | 5′-TTGGTGCAACTTAGCTGATC-3′ |

Sequences 1 through 10 respectively correspond to SEQ ID NOs: 5 through 14 shown in the below-described sequence listing.

Nucleotide Sequencing

The resultant PCR product was inserted into T-vector (product of Novagen), and the resultant vector was transfected into Escherichia coli strain JM109, followed by cloning of the Escherichia coli strain. Plasmid was extracted from the thus-cloned Escherichia coli, and the nucleotide sequence of fragments of the PCR product was determined by means of, for example, direct sequencing.

Extraction of Genomic DNA and PCR

Genomic DNA was extracted from peripheral blood lymphocytes by means of a customary technique. By use of the genomic DNA (serving as a template) and a PCR primer shown in Table 2, intron 1, intron 4, and intron 6 regions of an IL-12.p40 subunit gene were amplified under the same conditions as those of the above-described RT-PCR. The nucleotide sequence of fragments of the resultant PCR product was determined by means of direct sequencing.

TABLE 2

| Sequence | Intron | Primer sequence |
|---|---|---|
| 11 | IL-12 · p40 intron 1 | 5′-GGCTTAAAGGGGCCAAGT-3′ |
| 12 | IL-12 · p40 intron 1 | 5′-AGGGAGCACTATCCCTCAGC-3′ |
| 13 | IL-12 · p40 intron 4 | 5′-GTCCAGAGACATGTAAGTGC-3′ |
| 14 | IL-12 · p40 intron 4 | 5′-GAGATGATGCTTGTCAACCA-3′ |
| 15 | IL-12 · p40 intron 6 | 5′-GCTAGAAAGATGAAAGCTGG-3′ |
| 16 | IL-12 · p40 intron 6 | 5′-GTTTCTGATTCTGGCAACTG-3′ |

Sequences 11 through 16 respectively correspond to SEQ ID NOs: 15 through 20 shown in the below-described sequence listing.

[Clinical Analysis]

Peripheral blood mononuclear cells were isolated from 75 subjects with allergic (atopic) conditions (hereinafter the group of the subjects may be referred to as “allergic group”) and from 62 healthy subjects complaining of no allergic (atopic) symptoms (i.e., control) (hereinafter the group of the healthy subjects may be referred to as “non-allergic group”). The thus-isolated cells were stimulated with IL-12 or PHA, and the amount of produced IFN-γ was measured.

In the non-allergic group, the amount of IFN-γ produced through PHA stimulation was found to be 116 to 10,338 pg/ml (average: 2,886 pg/ml), whereas in the allergic group, the amount of IFN-γ produced through PHA stimulation was found to be reduced to 46 to 10,000 pg/ml (average: 1,324 pg/ml). In the non-allergic group, the amount of IFN-γ produced through IL-12 stimulation was found to be 55 to 10,000 pg/ml (average: 971 pg/ml), whereas in the allergic group, the amount of IFN-γ produced through IL-12 stimulation was found to be significantly reduced to the detection limit or less to 1,130 pg/ml (average: 145 pg/ml). In twenty-four of the 75 subjects with allergic (atopic) conditions, the amount of IFN-γ produced through IL-12 stimulation was found to be the detection limit or less. In each of the subjects with allergic (atopic) conditions in whom the amount of produced IFN-γ was found to be the detection limit or less, detection of an IL-12R β2 chain gene was performed. As a result, the following gene polymorphisms were identified.

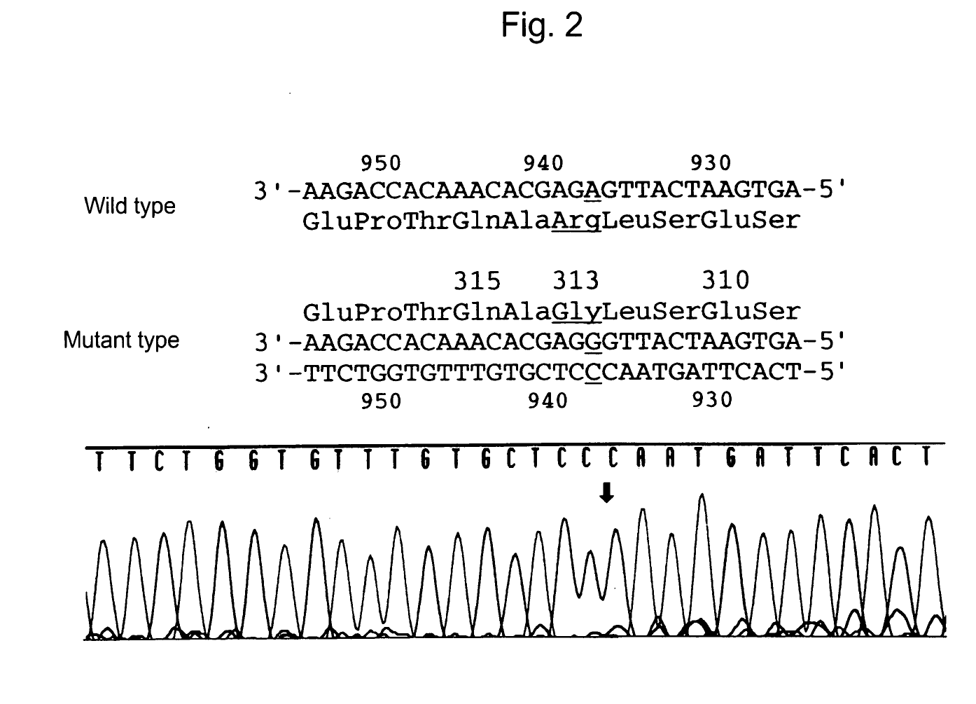

Gene Polymorphism 1 (see FIG. 2)

As is clear from the nucleotide sequence, electropherogram, and analysis data shown in FIG. 2, gene polymorphism 1 is such that the wild-type base (A) at position 937 of the IL-12R β2 chain gene is substituted by G, whereby the wild-type amino acid residue (Arg (AGA)) at position 313 of the IL-12R β2 chain protein is substituted by Gly (GGA) (i.e., gene polymorphism 1 is represented as R313G polymorphism).

As described below, it has been elucidated that gene polymorphism 1—which is found at a region encoding position 313 Arg of the IL-12R β2 chain protein encoded by the IL-12R β2 chain gene—relates to an allergic predisposition of a subject.

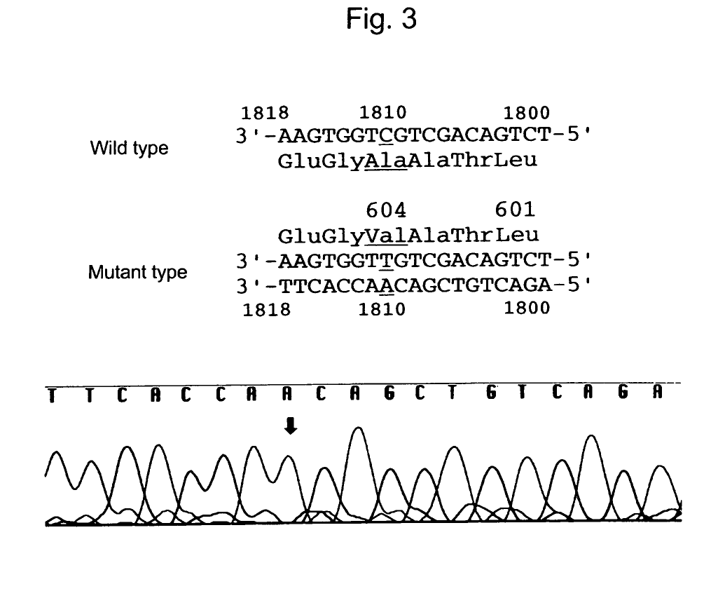

Gene Polymorphism 2 (see FIG. 3)

As is clear from the nucleotide sequence, electropherogram, and analysis data shown in FIG. 3, gene polymorphism 2 is such that the wild-type base (C) at position 1,811 of the IL-12R β2 chain gene is substituted by T, whereby the wild-type amino acid residue (Ala (GCT)) at position 604 of the IL-12R β2 chain protein is substituted by Val (GTT) (i.e., gene polymorphism 2 is represented as A604V polymorphism).

As described below, it has been elucidated that gene polymorphism 2—which is found at a region encoding position 604 Ala of the IL-12R β2 chain protein encoded by the IL-12R β2 chain gene—relates to an allergic predisposition of a subject.

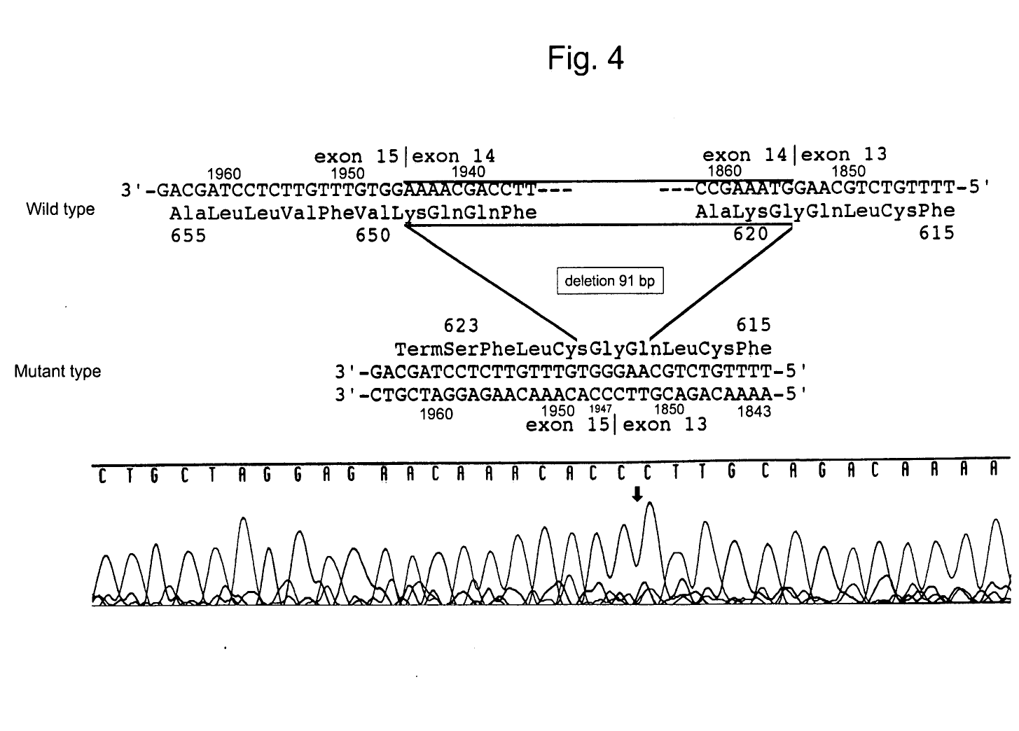

Gene Polymorphism 3 (see FIG. 4)

As is clear from the nucleotide sequence, electropherogram, and analysis data shown in FIG. 4, gene polymorphism 3 is specified such that frameshift occurs as a result of deletion of 91 bases at positions 1,856 through 1,946 of the wild-type IL-12R β2 chain gene, whereby the amino acid residue located at the 45th position on the downstream side of the deleted site is substituted by a termination codon (TAG) (i.e., gene polymorphism 3 is represented as 1856de191 polymorphism). It is expected that the 91 base deletion causes production of a mutant IL-12R β2 chain protein having 623 amino acid residues.

As described below, it has been elucidated that gene polymorphism 3—which is specified by lack of position 619 glycine and subsequent amino acid residues of the IL-12R β2 chain protein encoded by the IL-12R β2 chain gene—relates to an allergic predisposition of a subject.

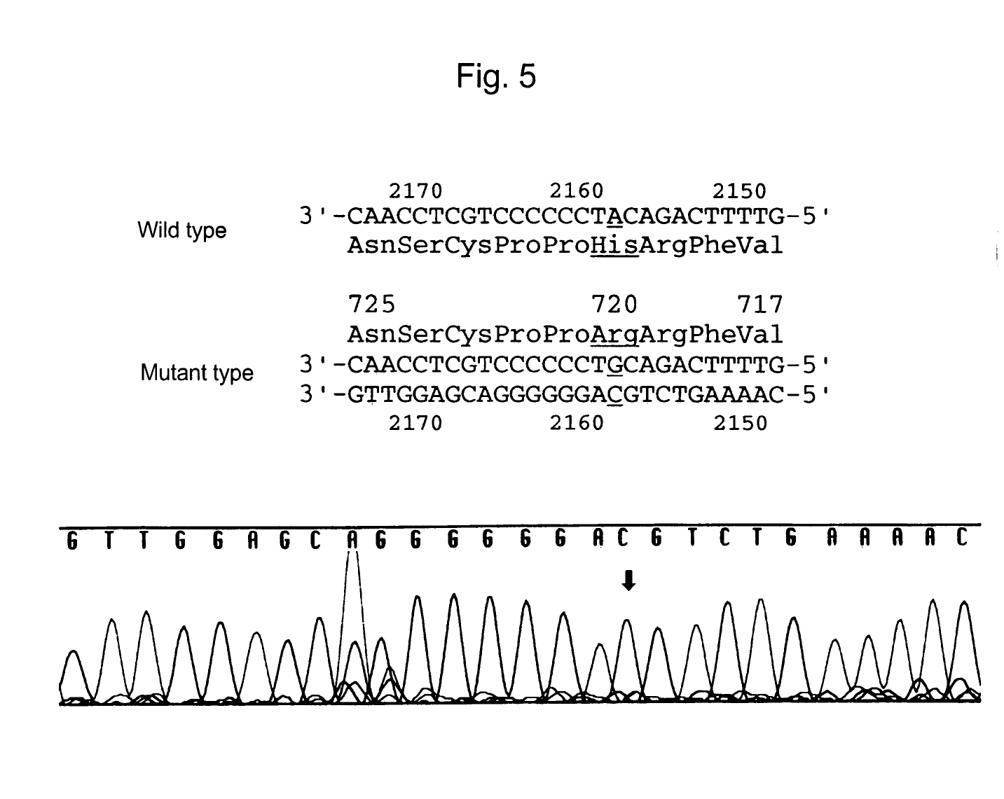

Gene Polymorphism 4 (see FIG. 5)

As is clear from the nucleolide sequence, electropherogram, and analysis data shown in FIG. 5, gene polymorphism 4 is such that the wild-type base (A) at position 2,159 of the IL-12R β2 chain gene is substituted by G, whereby the wild-type amino acid residue (His (CAT)) at position 720 of the IL-12R β2 chain protein is substituted by Arg (CGT) (i.e., gene polymorphism 4 is represented as H720R polymorphism).

As described below, it has been elucidated that gene polymorphism 4—which is found at a region encoding position 720 His of the IL-12R β2 chain protein encoded by the IL-12R β2 chain gene—relates to an allergic predisposition of a subject.

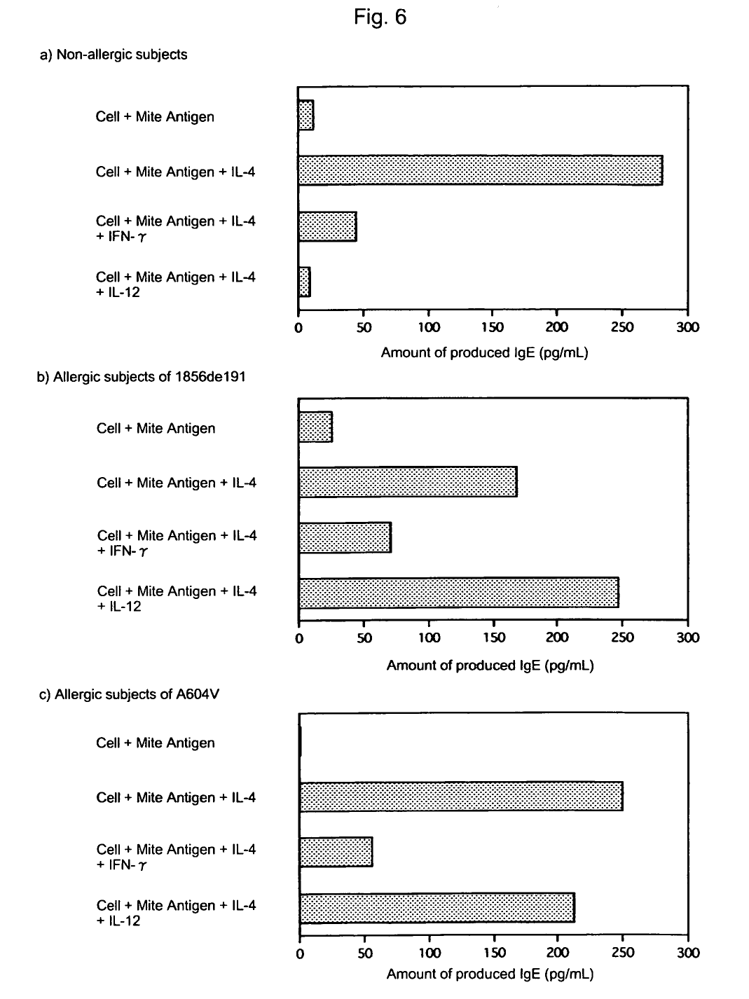

In a manner similar to that as described above, peripheral blood mononuclear cells were isolated from subjects who were found to have gene polymorphisms 1 through 4. The thus-isolated cells were cultured in the presence of IL-4, IFN-γ, or IL-12, and the amount of IgE secreted in the resultant culture supernatant was measured. As a result, in mononuclear cells derived from subjects with allergic (atopic) conditions who were not found to have such a polymorphic base or amino acid residue, IgE production, which is induced by IL-4, was suppressed when IL-4 coexisted with IFN-γ or IL-12. In contrast, in mononuclear cells derived from subjects with allergic (atopic) conditions who were found to have such a polymorphic base or amino acid residue, when IL-4 coexisted with IFN-γ, IgE production was suppressed, whereas when IL-4 coexisted with IL-12, IgE production was not suppressed (FIG. 6). The results indicate that abnormality occurs in the signal transduction system of IL-12-mediated IFN-γ production, and thus IFN-γ is insufficiently produced, and this is attributed to the functional disorder of IL-12R.

The above results show that gene polymorphisms 1 through 4 cause the functional disorder of IL-12R, resulting in insufficient production of IFN-γ, and thus IgE production is not sufficiently suppressed by IFN-γ. Gene polymorphisms 1 through 4 were found to be expressed in the subjects with allergic (atopic) conditions at a significantly high frequency (P=0.0179).

Polymorphisms 1, 3, and 4 of the IL-12R β2 chain gene were detected in ten subjects (eight subjects with 1856de191 polymorphism, one subject with R313G polymorphism, and one subject with H720R polymorphism) of 24 subjects with allergic (atopic) diseases in whom the amount of IFN-γ produced through IL-12 stimulation was found to be the detection limit or less, whereas the polymorphic bases or amino acid residues of these polymorphisms were not detected in the 62 healthy subjects (Table 3). Meanwhile, the A604V polymorphism was found to be expressed in subjects with allergic (atopic) conditions at a significantly high frequency (P<0.001) (Table 4).

TABLE 3

| | | Heterozygote | | | |
|---|---|---|---|---|---|
| | | R313G | 1856del91 | H720R | |
| | | poly- | poly- | poly- | |
| Target | Number | morphism | morphism | morphism | Total |
| Non-allergic | 62 | 0 | 0 | 0 | 0 |
| group | | | | | |
| Allergic group | 75 | 1 | 8 | 1 | 10 |
| Amount of | | | | | |
| produced IFN-γ | | | | | |
| <20 pg/ml | 24 | 1 | 8 | 1 | 10 |
| ≧20 pg/ml | 51 | 0 | 0 | 0 | 0 |

TABLE 4

| Genotype | Non-allergic group | Allergic group | Total |
|---|---|---|---|
| A604V | (n = 104) | (n = 102) | (n = 206) |
| C/C | 100 | 81 | 181 |
| T/C | 4 | 21* | 25 |

*P < 0.001

In addition, the following polymorphisms 5 through 7 of an IL-12R β1 chain gene were found to exist (see Table 5).

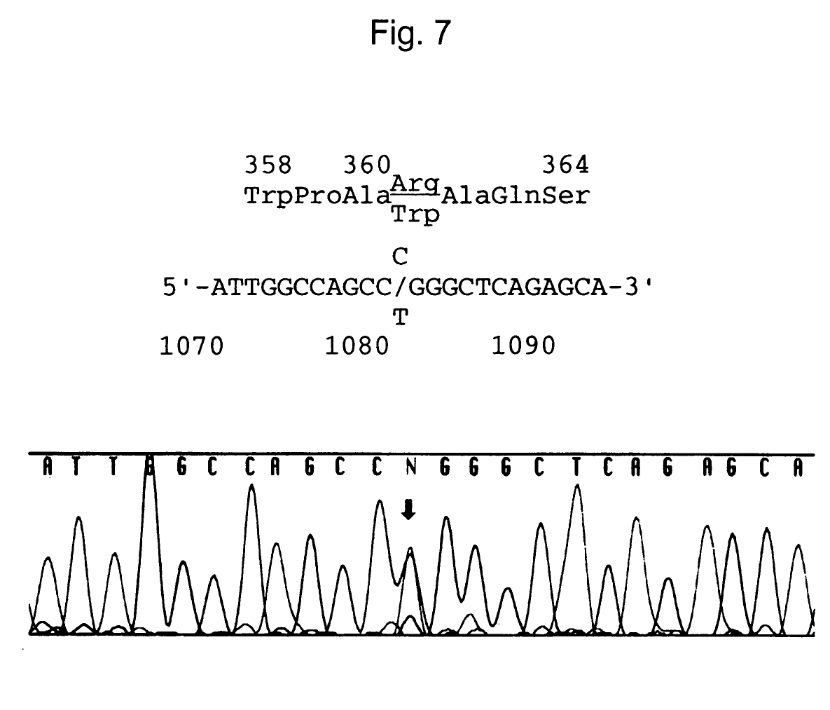

Gene Polymorphism 5 (see FIG. 7)

As is clear from the nucleotide sequence, electropherogram, and analysis data shown in FIG. 7, gene polymorphism 5 is such that the wild-type base (C) at position 1,081 of the IL-12R β1 chain gene is substituted by T, whereby the wild-type amino acid residue (Arg (CGG)) at position 361 of the IL-12R β1 chain protein is substituted by Trp (TGG) (i.e., gene polymorphism 5 is represented as R361W polymorphism).

As described below, it has been elucidated that gene polymorphism 5—which is found at a region encoding position 361 Arg of the IL-12R β1 chain protein encoded by the IL-12R β1 chain gene—relates to an allergic predisposition of a subject.

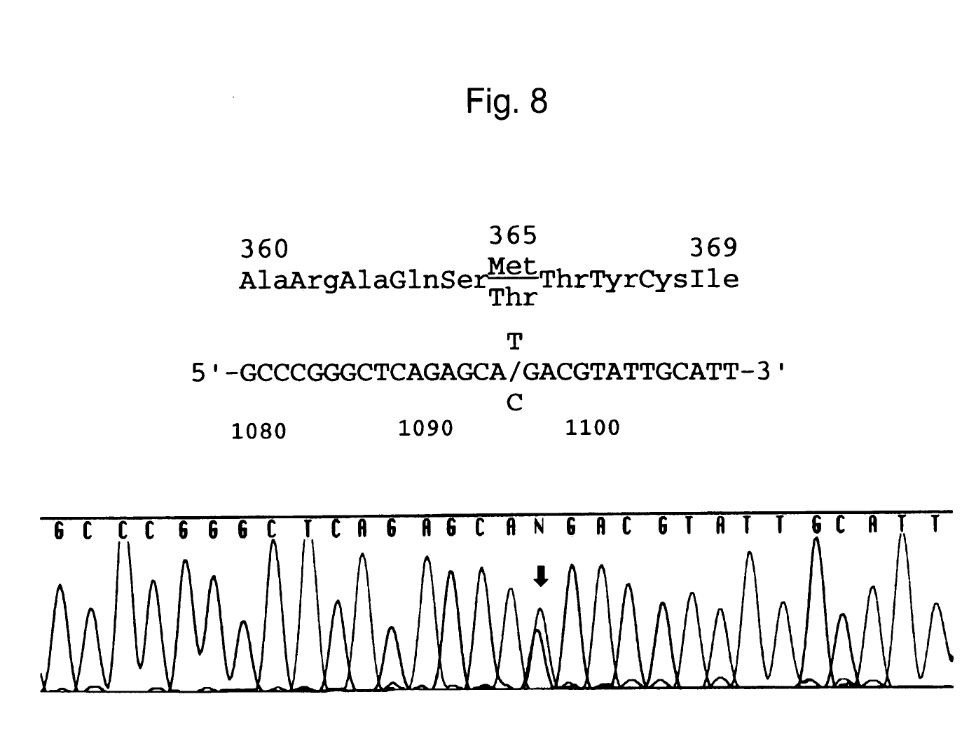

Gene Polymorphism 6 (see FIG. 8)

As is clear from the nucleotide sequence, electropherogram, and analysis data shown in FIG. 8, gene polymorphism 6 is such that the wild-type base (T) at position 1,094 of the IL-12R β1 chain gene is substituted by C, whereby the wild-type amino acid residue (Met (ATG)) at position 365 of the IL-12R β1 chain protein is substituted by Thr (ACG) (i.e., gene polymorphism 6 is represented as M365T polymorphism).

As described below, it has been elucidated that gene polymorphism 6—which is found at a region encoding position 365 methionine of the IL-12R β1 chain protein encoded by the IL-12R β1 chain gene—relates to an allergic predisposition of a subject.

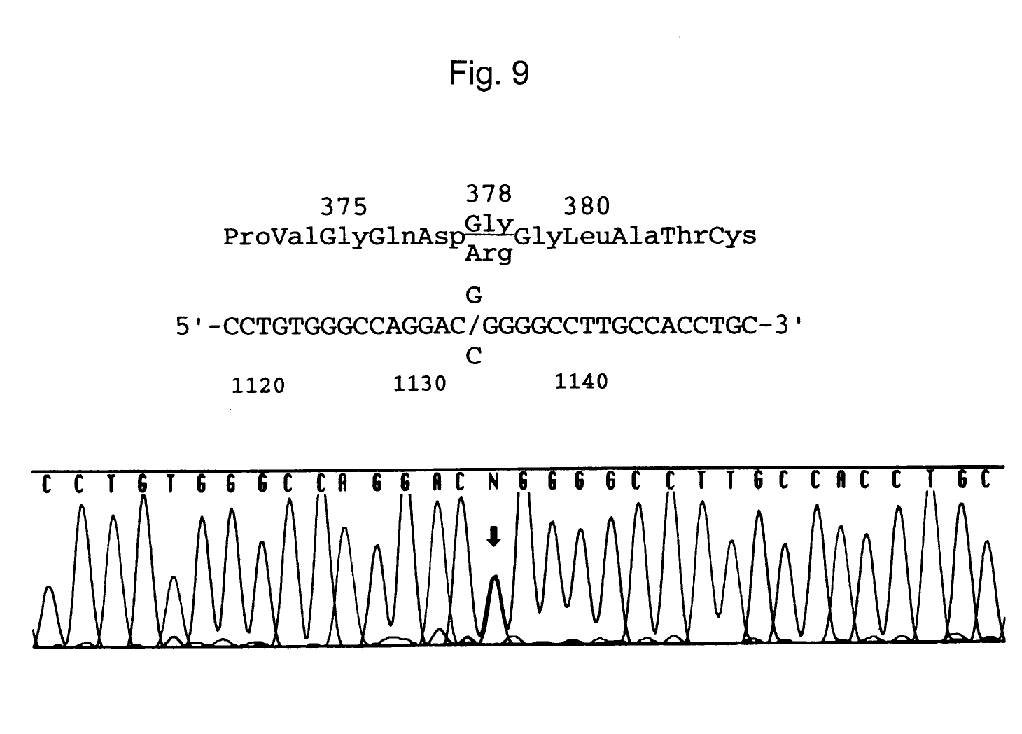

Gene Polymorphism 7 (see FIG. 9)

As is clear from the nucleotide sequence, electropherogram, and analysis data shown in FIG. 9, gene polymorphism 7 is such that the wild-type base (G) at position 1,132 of the IL-12R β1 chain gene is substituted by C, whereby the wild-type amino acid residue (Gly (GGG)) at position 378 of the IL-12R β1 chain protein is substituted by Arg (CGG) (i.e., gene polymorphism 7 is represented as G378R polymorphism).

As described below, it has been elucidated that gene polymorphism 7—which is found at a region encoding position 378 glycine of the IL-12R β1 chain protein encoded by the IL-12R β1 chain gene—relates to an allergic predisposition of a subject.

Table 5 shows the frequency of expression of the aforementioned three polymorphisms of the IL-12R β1 chain gene. These gene polymorphisms containing a polymorphic base or amino acid residue were found to be expressed at a high frequency in subjects with allergic (atopic) conditions, as compared with the case of healthy subjects.

TABLE 5

| | Non-allergic group | Allergic group |
|---|---|---|
| Genotype | (n = 50) | (n = 45) |
| R361W | | |
| C/C | 50 | 44 |
| T/C | 0 | 1 |
| T/T | 0 | 0 |
| M365T | | |
| T/T | 33 | 22 |
| C/T | 5 | 8 |
| C/C | 12 | 15 |
| G378R | | |
| G/G | 33 | 22 |
| C/G | 5 | 8 |
| C/C | 12 | 15 |

Furthermore, the following gene polymorphism relating to an allergic predisposition was newly detected through analysis of IL-18R α chain gene polymorphisms.

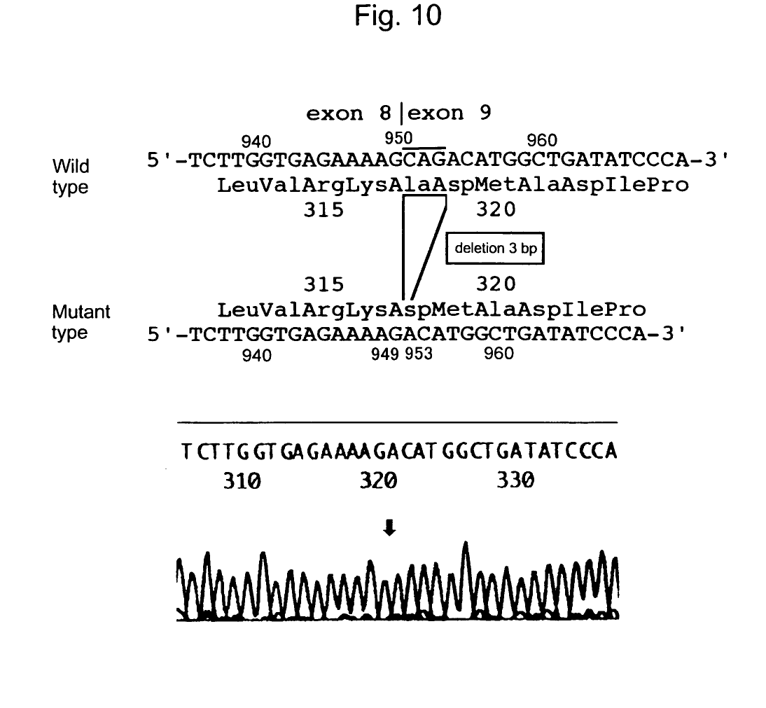

Gene Polymorphism 8 (see FIG. 10)

As is clear from the nucleotide sequence, electropherogram, and analysis data shown in FIG. 10, gene polymorphism 8 is specified by deletion of three bases (C, A, and G) at positions 950 through 952 of a wild-type IL-18R α chain gene (i.e., gene polymorphism 8 is represented as 950de13 polymorphism). This polymorphism is considered to cause synthesis of a mutant protein containing 540 amino acid residues; i.e., a protein which does not contain the position 317 amino acid residue (Ala) of a wild-type IL-18R α chain protein. Also, this polymorphism is considered to be generated through alternative splicing, since it is not detected in the nucleotide sequence of genomic DNA.

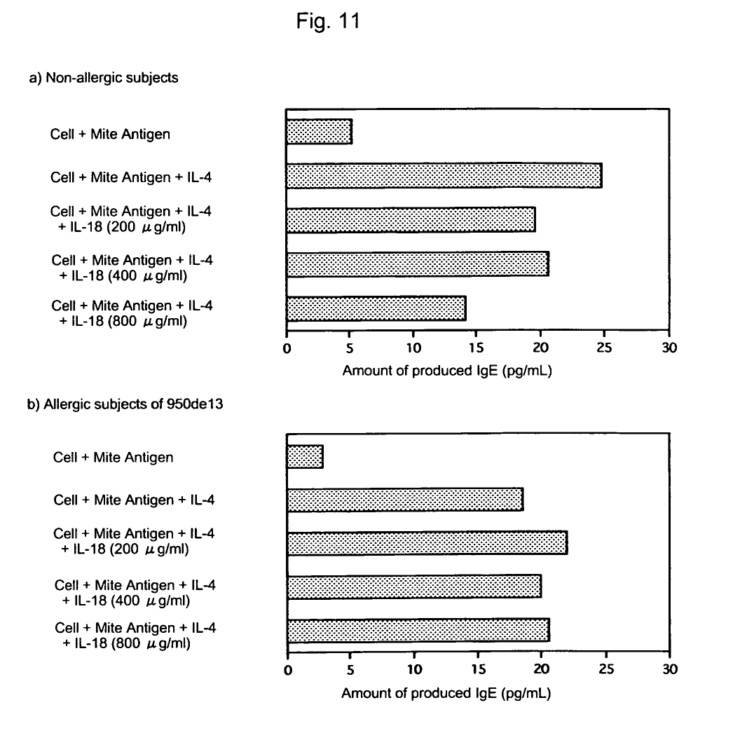

In a manner similar to that as described above, peripheral blood mononuclear cells were isolated from subjects with allergic (atopic) conditions who were found to have a polymorphic base or amino acid residue of gene polymorphism 8 and from subjects with allergic (atopic) conditions who were not found to have a polymorphic base or amino acid residue of gene polymorphism 8. The thus-isolated cells were cultured in the presence of IL-4 or IL-18, and the amount of IgE secreted in the resultant culture supernatant was measured. As a result, in mononuclear cells derived from the subjects with allergic (atopic) conditions who were not found to have a polymorphic base or amino acid residue of gene polymorphism 8, IL-4-induced IgE production was observed to be suppressed in an IL-18-concentration-dependent manner, whereas in mononuclear cells derived from the subjects who were found to have a polymorphic base or amino acid residue of gene polymorphism 8, suppression of IgE production by IL-18 was not observed (FIG. 11).

The results indicate that, in the subjects who are found to have a polymorphic base or amino acid residue of gene polymorphism 8, IgE production is not suppressed by IFN-γ produced through the IL-18-mediated signal transduction; i.e., IL-18R has any functional disorder. The above results show that the polymorphic base or amino acid residue of gene polymorphism 8 causes the functional disorder of IL-18R, resulting in no production of IFN-γ, and thus IgE production is not suppressed by IFN-γ. Gene polymorphism 8 was found to be expressed in subjects with allergic (atopic) conditions at a significantly high frequency (P−0.0179) (Table 6).

TABLE 6

| Genotype | Non-allergic group | Allergic group | Total |
|---|---|---|---|
| 950de13 | (n = 41) | (n = 39) | (n = 80) |
| wild/wild | 9 | 4* | 13 |
| del3/wild | 30 | 26* | 56 |
| del3/del3 | 2 | 9* | 11 |

*P = 0.0179

Furthermore, the following gene polymorphism relating to an allergic predisposition was newly detected through analysis of IFN-γR 1 chain gene polymorphisms.

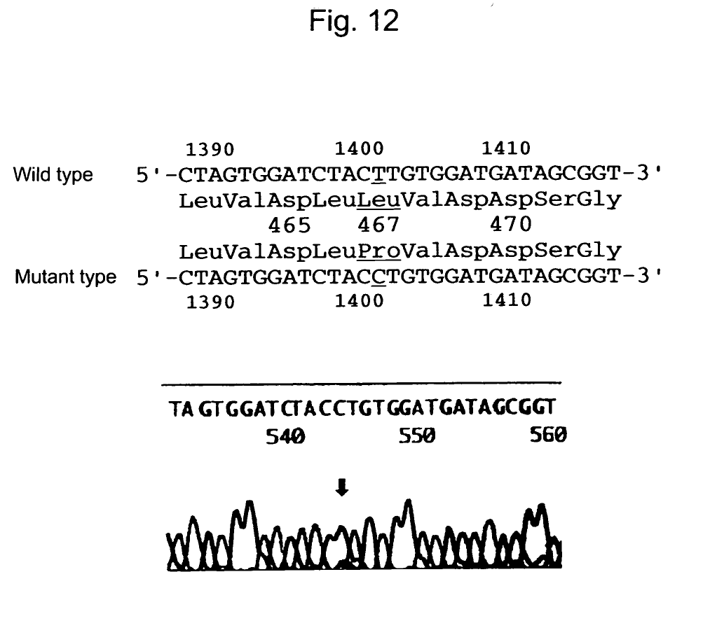

Gene Polymorphism 9 (see FIG. 12)

As is clear from the nucleotide sequence, electropherogram, and analysis data shown in FIG. 12, gene polymorphism 9 is such that the wild-type base (I) at position 1,400 of the IFN-γR 1 chain gene is substituted by C, whereby the wild-type amino acid residue (Leu (CTT)) at position 467 of the IFN-γR 1 chain protein is substituted by Pro (CCT) (i.e., gene polymorphism 9 is represented as L467P polymorphism). Since the L467P polymorphism is generated in the vicinity of an STAT1 binding site, conceivably, binding of STAT1, which is an important signal transduction molecule, fails to occur because of the presence of a polymorphic base or amino acid residue of gene polymorphism 9, and the signal transduction is suppressed.

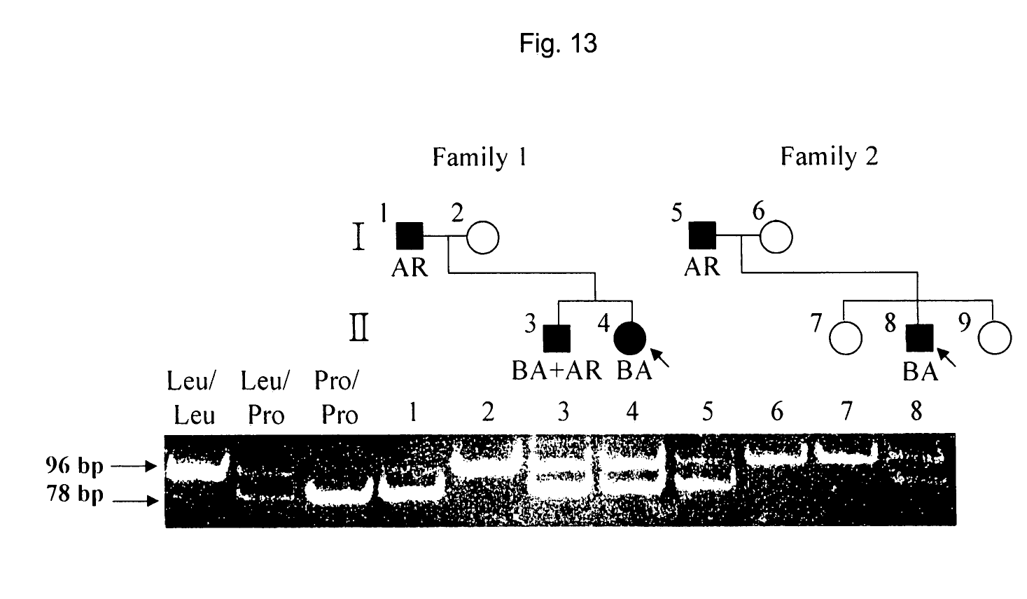

Through pedigree analysis in relation to gene polymorphism 9, a polymorphic base or amino acid residue of gene polymorphism 9 was detected only in families with bronchial asthma or atopic dermatitis. Therefore, the L467P polymorphism (i.e., a gene polymorphism specified by mutation of a site encoding position 467 leucine of the IFN-γR 1 chain protein) is considered to be specific to bronchial asthma or atopic dermatitis (FIG. 13: “BA” and “AR” shown in FIG. 13 represent bronchial asthma and allergic rhinitis, respectively). Studies were performed on the L467P polymorphism in 114 subjects with allergic (atopic) conditions and 102 non-allergic (non-atopic) subjects, and as a result, a polymorphic base or amino acid residue of gene polymorphism 9 was detected only in six subjects with allergic (atopic) conditions (2.8%).

The results show that the L467P polymorphism is one of the causes of allergic (atopic) diseases (Table 7).

TABLE 7

| Genotype | Non-allergic group | Allergic group | Total | |
|---|---|---|---|---|
| L467P | (n = 102) | (n = 114) | (n = 216) | |
| T/T | 102 | 108* | 210 | (97.2%) |
| C/T | 0 | 6* | 6 | (2.8%) |
| C/C | 0 | 0 | 0 | (0%) |

*P = 0.033

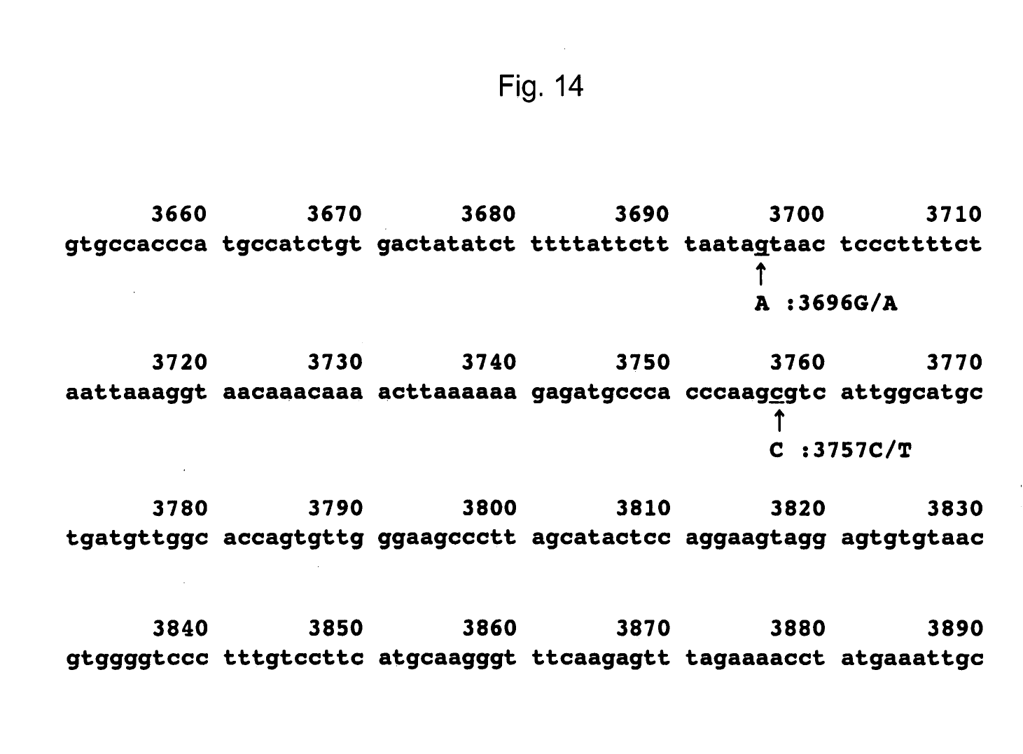

Gene Polymorphisms 10 and 11 (see FIG. 14)

Gene polymorphism 10 is specified by guanine (wild-type base) and adenine (polymorphic base) which are located at position 3,696 of the nucleotide sequence of the genomic gene encoding IL-12.p40; the guanine and adenine being located in intron 1 of the gene (i.e., gene polymorphism 10 is represented as 3696G/A). Gene polymorphism 11 is specified by cytosine (wild-type base) and thymine (polymorphic base) which are located at position 3,757 of the nucleotide sequence of the genomic gene; the cytosine and thymine being located in intron 1 of the gene (i.e., gene polymorphism 11 is represented as 3757C/T).

The frequency of expression of these gene polymorphisms was determined in an allergic group (33 subjects) and a non-allergic group (33 subjects). As shown in Table 8, in the allergic group, the A allele of gene polymorphism 10 was found to be expressed at a significantly high frequency (P=0.0146).

As shown in Table 9, in the allergic group, the T allele of gene polymorphism 11 was found to be expressed at a significantly high frequency (P=0.0146).

The analysis results of gene polymorphism 10 are the same as those of gene polymorphism 11, which indicates that these gene polymorphisms are in linkage equilibrium with each other.

TABLE 8

| | Non-allergic group | Allergic group | Total |
|---|---|---|---|
| Genotype | (n = 33) | (n = 33) | (n = 66) |
| 3696G/A | | | |
| G/G | 12 | 5 | 17 |
| A/G | 14 | 14 | 28 |
| A/A | 7 | 14 | 21 |
| Allele expression | | | |
| frequency | | | |
| G allele | 0.567 | 0.367 | |
| A allele | 0.424 | 0.638 | P = 0.0146 |

TABLE 9

| | Non-allergic group | Allergic group | Total |
|---|---|---|---|
| Genotype | (n = 33) | (n = 33) | (n = 66) |
| 3757C/T | | | |
| C/C | 12 | 5 | 17 |
| T/C | 14 | 14 | 28 |
| T/T | 7 | 14 | 21 |
| Allele expression | | | |
| frequency | | | |
| C allele | 0.567 | 0.367 | |
| T allele | 0.424 | 0.638 | P = 0.0146 |

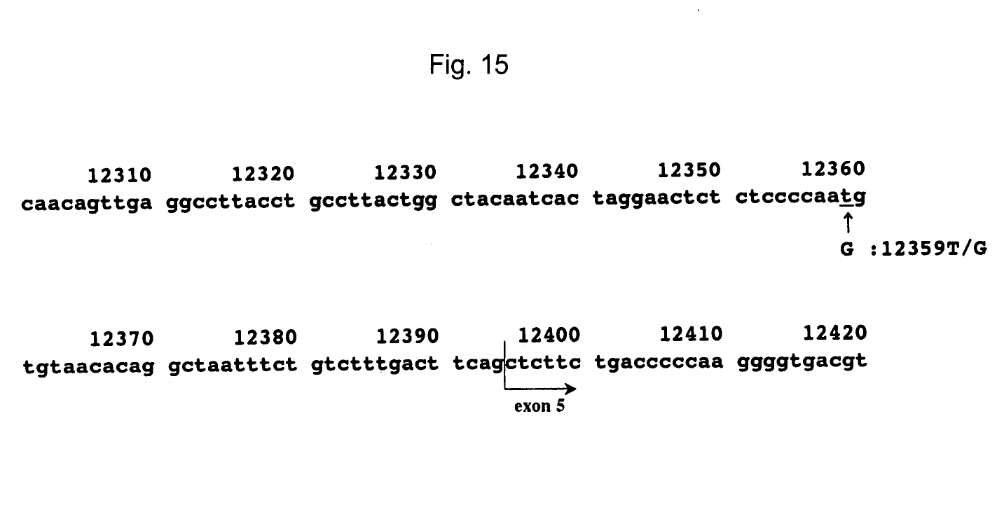

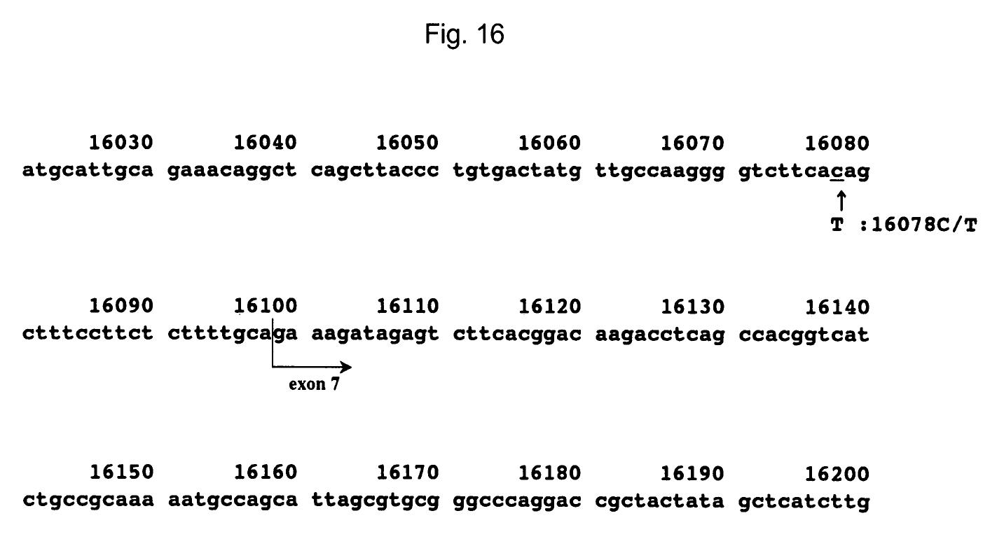

Gene Polymorphisms 12 and 13 [see FIG. 15 (Polymorphism 12) and FIG. 16 (Polymorphism 13)]

Gene polymorphism 12 is specified by thymine (wild-type base) and guanine (polymorphic base) which are located at position 12,359 of the nucleotide sequence of the genomic gene encoding IL-12.p40; the thymine and guanine being located in intron 4 of the gene (i.e., gene polymorphism 12 is represented as 12359T/G). Gene polymorphism 13 is specified by cytosine (wild-type base) and thymine (polymorphic base) which are located at position 16,078 of the nucleotide sequence of the genomic gene; the cytosine and thymine being located in intron 6 of the gene (i.e., gene polymorphism 13 is represented as 16078C/T).

The frequency of expression of these gene polymorphisms was determined in an allergic group (41 subjects) and a non-allergic group (57 subjects). As shown in Table 10, in the allergic group, the G allele of gene polymorphism 12 was found to be expressed at a very high frequency (P=0.06495).

As shown in Table 11, in the allergic group, the T allele of gene polymorphism 13 was found to be expressed at a very high frequency (P=0.06495).

The analysis results of gene polymorphism 12 are the same as those of gene polymorphism 13, which indicates that these gene polymorphisms are in linkage equilibrium with each other.

TABLE 10

| | Non-allergic group | Allergic group | Total |
|---|---|---|---|
| Genotype | (n = 41) | (n = 57) | (n = 98) |
| 12359T/G | | | |
| T/T | 35 | 48 | 83 |
| G/T | 6 | 8 | 14 |
| G/G | 0 | 1 | 1 |
| Allele expression | | | |
| frequency | | | |
| T allele | 0.927 | 0.912 | |
| G allele | 0.073 | 0.088 | P = 0.06495 |

TABLE 11

| | Non-allergic group | Allergic group | Total |
|---|---|---|---|
| Genotype | (n = 41) | (n = 57) | (n = 98) |
| 16078C/T | | | |
| C/C | 35 | 48 | 83 |
| T/C | 6 | 8 | 14 |
| T/T | 0 | 1 | 1 |
| Allele expression | | | |
| frequency | | | |
| C allele | 0.927 | 0.912 | |
| T allele | 0.073 | 0.088 | P = 0.06495 |

### INDUSTRIAL APPLICABILITY

The present invention provides a gene detection method which enables detection of an allergic predisposition, by using a gene polymorphism relating to allergy as an index.

1. A gene detection method comprising detecting one or more gene polymorphisms selected from the group consisting of the below-described gene polymorphisms (a) through (m), to thereby detect an allergic predisposition of a subject:
(a) a polymorphism of an interleukin 12 receptor β2 chain gene specified by mutation of a region encoding position 313 arginine of an interleukin 12 receptor β2 chain protein encoded by the gene;
(b) a polymorphism of an interleukin 12 receptor β2 chain gene specified by mutation of a region encoding position 604 alanine of an interleukin β2 receptor β2 chain protein encoded by the gene;
(c) a polymorphism of an interleukin 12 receptor β2 chain gene specified by lack of a region encoding position 619 glycine and subsequent amino acid residues of an interleukin 12 receptor β2 chain protein encoded by the gene;
(d) a polymorphism of an interleukin 12 receptor β2 chain gene specified by mutation of a region encoding position 720 histidine of an interleukin 12 receptor β2 chain protein encoded by the gene;
(e) a polymorphism of an interleukin 12 receptor β1 chain gene specified by mutation of a region encoding position 361 arginine of an interleukin 12 receptor β1 chain protein encoded by the gene;
(f) a polymorphism of an interleukin 12 receptor β1 chain gene specified by mutation of a region encoding position 365 methionine of an interleukin 12 receptor β1 chain protein encoded by the gene;
(g) a polymorphism of an interleukin 12 receptor β1 chain gene specified by mutation of a region encoding position 378 glycine of an interleukin 12 receptor β1 chain protein encoded by the gene;
(h) a polymorphism of an interleukin 18 receptor α chain gene specified by lack of position 317 alanine of an interleukin 18 receptor α chain protein encoded by the gene;
(i) a polymorphism of an interferon γ receptor 1 chain gene specified by mutation of a region encoding position 467 leucine of an interferon γ receptor 1 chain protein encoded by the gene;
(j) a polymorphism of an interleukin 12.p40 subunit gene specified by substitution of position 3,696 guanine by another base, the guanine being in intron 1 of the gene;
(k) a polymorphism of an interleukin 12.p40 subunit gene specified by substitution of position 3,757 cytosine by another base, the cytosine being in intron 1 of the gene;
(l) a polymorphism of an interleukin 12.p40 subunit gene specified by substitution of position 12,359 thymine by another base, the thymine being in intron 4 of the gene; and
(m) a polymorphism of an interleukin 12.p40 subunit gene specified by substitution of position 16,078 cytosine by another base, the cytosine being in intron 6 of the gene.

2. The gene detection method according to claim 1, wherein said gene polymorphism (a), which is specified by mutation of a region encoding position 313 arginine of an interleukin 12 receptor β2 chain protein encoded by an interleukin 12 receptor β2 chain gene, is specified such that the position 937 polymorphic base of the interleukin 12 receptor β2 chain gene is guanine, and the position 313 polymorphic amino acid residue of the interleukin 12 receptor β2 chain protein is glycine.

3. The gene detection method according to claim 1, wherein said gene polymorphism (b), which is specified by mutation of a region encoding position 604 alanine of an interleukin 12 receptor β2 chain protein encoded by an interleukin 12 receptor β2 chain gene, is specified such that the position 1,811 polymorphic base of the interleukin 12 receptor β2 chain gene is thymine, and the position 604 polymorphic amino acid residue of the interleukin 12 receptor β2 chain protein is valine.

4. The gene detection method according to claim 1, wherein said gene polymorphism (c), which is specified by lack of a region encoding position 619 glycine and subsequent amino acid residues of an interleukin 12 receptor β2 chain protein encoded by an interleukin 12 receptor β2 chain gene, is specified by lack of the bases at positions 1,856 to 1,946 of the interleukin 12 receptor β2 chain gene.

5. The gene detection method according to claim 1, wherein said gene polymorphism (d), which is specified by mutation of a region encoding position 720 histidine of an interleukin 12 receptor β2 chain protein encoded by an interleukin 12 receptor β2 chain gene, is specified such that the position 2,159 polymorphic base of the interleukin 12 receptor β2 chain gene is guanine, and the position 720 polymorphic amino acid residue of the interleukin 12 receptor β2 chain protein is arginine.

6. The gene detection method according to claim 1, wherein said gene polymorphism (e), which is specified by mutation of a region encoding position 361 arginine of an interleukin 12 receptor β1 chain protein encoded by an interleukin 12 receptor β1 chain gene, is specified such that the position 1,081 polymorphic base of the interleukin 12 receptor β1 chain gene is thymine, and the position 361 polymorphic amino acid residue of the interleukin 12 receptor β1 chain protein is tryptophan.

7. The gene detection method according to claim 1, wherein said gene polymorphism (f), which is specified by mutation of a region encoding position 365 methionine of an interleukin 12 receptor β1 chain protein encoded by an interleukin 12 receptor β1 chain gene, is specified such that the position 1,094 polymorphic base of the interleukin 12 receptor β1 chain gene is cytosine, and the position 365 polymorphic amino acid residue of the interleukin 12 receptor β1 chain protein is threonine.

8. The gene detection method according to claim 1, wherein said gene polymorphism (g), which is specified by mutation of a region encoding position 378 glycine of an interleukin 12 receptor β1 chain protein encoded by an interleukin 12 receptor β1 chain gene, is specified such that the position 1,132 polymorphic base of the interleukin 12 receptor β1 chain gene is cytosine, and the position 378 polymorphic amino acid residue of the interleukin 12 receptor β1 chain protein is arginine.

9. The gene detection method according to claim 1, wherein said gene polymorphism (h), which is specified by lack of position 317 alanine of an interleukin 18 receptor α chain protein encoded by an interleukin 18 receptor α chain gene, is specified by lack of the bases at positions 950 to 952 of the interleukin 18 receptor α chain gene.

10. The gene detection method according to claim 1, wherein said gene polymorphism (i), which is specified by mutation of a region encoding position 467 leucine of an interferon γ receptor 1 chain protein encoded by an interferon γ receptor 1 chain gene, is specified such that the position 1,400 polymorphic base of the interferon γ receptor 1 chain gene is cytosine, and the position 467 polymorphic amino acid residue of the interferon γ receptor 1 chain protein is proline.

11. The gene detection method according to claim 1, wherein said gene polymorphism (j) is specified such that the position 3,696 polymorphic base of the interleukin 12.p40 subunit gene, which base is in intron 1 of the gene, is adenine.

12. The gene detection method according to claim 1, wherein said gene polymorphism (k) is specified such that the position 3,757 polymorphic base of the interleukin 12.p40 subunit gene, which base is in intron 1 of the gene, is thymine.

13. The gene detection method according to claim 1, wherein said gene polymorphism (l) is specified such that the position 12,359 polymorphic base of the interleukin 12.p40 subunit gene, which base is in intron 4 of the gene, is guanine.

14. The gene detection method according to claim 1, wherein said gene polymorphism (m) is specified such that the position 16,078 polymorphic base of the interleukin 12.p40 subunit gene, which base is in intron 6 of the gene, is thymine.

15. The gene detection method according to claim 1, wherein, in the case of detection of a gene polymorphism in introns of the interleukin 12.p40 subunit gene, a polymorphism of the position 3,696 base or position 3,757 base is detected in intron 1 of the gene.

16. The gene detection method according to claim 1, wherein, in the case of detection of a gene polymorphism in introns of the interleukin 12.p40 subunit gene, a polymorphism of the position 12,359 base is detected in intron 4 of the gene, or a polymorphism of the position 16,078 base is detected in intron 6 of the gene.

17. The gene detection method according to claim 1, which employs the Invader assay for detecting a gene polymorphism.

18. A gene detection kit comprising an element for detecting one or more gene polymorphisms selected from the group consisting of the below-described gene polymorphisms (a) through (m):
(a) a polymorphism of an interleukin 12 receptor β2 chain gene specified by mutation of a region encoding position 313 arginine of an interleukin 12 receptor β2 chain protein encoded by the gene;
(b) a polymorphism of an interleukin 12 receptor β2 chain gene specified by mutation of a region encoding position 604 alanine of an interleukin 12 receptor β2 chain protein encoded by the gene;
(c) a polymorphism of an interleukin 12 receptor β2 chain gene specified by lack of a region encoding position 619 glycine and subsequent amino acid residues of an interleukin 12 receptor β2 chain protein encoded by the gene;
(d) a polymorphism of an interleukin 12 receptor β2 chain gene specified by mutation of a region encoding position 720 histidine of an interleukin 12 receptor β2 chain protein encoded by the gene;
(e) a polymorphism of an interleukin 12 receptor β1 chain gene specified by mutation of a region encoding position 361 arginine of an interleukin 12 receptor β1 chain protein encoded by the gene;
(f) a polymorphism of an interleukin 12 receptor β1 chain gene specified by mutation of a region encoding position 365 methionine of an interleukin 12 receptor β1 chain protein encoded by the gene;
(g) a polymorphism of an interleukin 12 receptor β1 chain gene specified by mutation of a region encoding position 378 glycine of an interleukin 12 receptor β1 chain protein encoded by the gene;
(h) a polymorphism of an interleukin 18 receptor a chain gene specified by lack of position 317 alanine of an interleukin 18 receptor α chain protein encoded by the gene;
(i) a polymorphism of an interferon γ receptor 1 chain gene specified by mutation of a region encoding position 467 leucine of an interferon γ receptor 1 chain protein encoded by the gene;
(j) a polymorphism of an interleukin 12.p40 subunit gene specified by substitution of position 3,696 guanine by another base, the guanine being in intron 1 of the gene;
(k) a polymorphism of an interleukin 12.p40 subunit gene specified by substitution of position 3,757 cytosine by another base, the cytosine being in intron 1 of the gene;
(l) a polymorphism of an interleukin 12.p40 subunit gene specified by substitution of position 12,359 thymine by another base, the thymine being in intron 4 of the gene; and
(m) a polymorphism of an interleukin 12.p40 subunit gene specified by substitution of position 16,078 cytosine by another base, the cytosine being in intron 6 of the gene.

19. The gene detection kit according to claim 18, which employs the Invader assay for detecting a gene polymorphism.
